(19) United States
(12) Patent Application Publication
(10) Pub. No.: US 2015/0168429 A1
(43) Pub. Date: Jun. 18, 2015

(54) Lp(a) SUBFORM SIZE IDENTIFICATION USING ZONAL GEL IMMUNO-FIXATION ELECTROPHORESIS

(71) Applicant: HEALTH DIAGNOSTIC LABORATORY, INC., Richmond, VA (US)

(72) Inventors: Philip GUADAGNO, Mechanicsville, VA (US); Erin Grace SUMMERS, Sandston, VA (US)

(21) Appl. No.: 14/575,836

(22) Filed: Dec. 18, 2014

Related U.S. Application Data
(60) Provisional application No. 61/917,823, filed on Dec. 18, 2013.
(60) Provisional application No. 62/005,658, filed on May 30, 2014.
(60) Provisional application No. 62/042,613, filed on Aug. 27, 2014.

(51) Int. Cl. G01N 33/92

(52) U.S. Cl. CPC G01N 33/92; G01N 2800/32; G01N 2800/52; G01N 2333/775

(57) ABSTRACT

In one aspect, a method for determining the composition of individual Lp(a) subforms in a test sample is provided. The method involves providing a test sample comprising Lp(a) subforms obtained from a subject; separating the Lp(a) subforms in the test sample along an electrophoretic gel; measuring the migration velocity of the individual Lp(a) subforms along the electrophoretic gel; comparing, based on said measuring, the migration velocity of the individual Lp(a) subforms to a reference value; and determining, based on said comparing, the molar mass of the individual Lp(a) subforms. Methods for predicting cardiovascular health are also provided.

This application claims priority benefit of U.S. Provisional Patent Application No. 61/917,823, filed Dec. 18, 2013, and U.S. Provisional Patent Application No. 62/005,658, filed May 30, 2014, and U.S. Provisional Patent Application No. 62/042,613, filed Aug. 27, 2014, each of which is hereby incorporated by reference in its entirety.

### TECHNOLOGICAL FIELD

Disclosed herein are methods relating to the identification of Lp(a) subform size using gel electrophoresis.

### BACKGROUND

Electrophoresis is a technique used to separate charged species on the basis of size, electric charge, and other physical properties. In electrophoresis, the charged species migrate through a conductive electrophoretic medium, which may be (but is not required to be) a gel, under the influence of an electric field. Activated electrodes located at either end of the electrophoretic medium provide the driving force for the migration. The properties of the molecules, including their charge and mass, determine how rapidly the electric field causes them to migrate through the electrophoretic medium.

Many important biological molecules, such as amino acids, peptides, proteins, nucleotides, and nucleic acids, possess ionizable groups. Because of these ionizable groups, at any given pH, many important biological molecules exist in solution as electrically charged species. The electrically charged species enable doctors and scientists to separate nucleic acids and proteins using electrophoresis.

Separation of molecules, biological or otherwise, using electrophoresis depends on various forces, including charge and mass. When a biological sample, such as a protein or DNA, is mixed in a buffer solution and applied to an electrophoretic medium, these two forces act together. Separation using electrophoresis is possible because the rate of molecular migration through the electric field depends on the strength of the field, the charge, size, and shape of the molecules, and the ionic strength and temperature of the buffer through which the molecules are moving. During electrophoresis, the applied electrical field causes the molecules to move through the pores of the electrophoretic medium based on the molecular charge. The electrical potential at one electrode repels the molecules while the potential at the other electrode simultaneously attracts the molecules. The frictional force of the electrophoretic medium also aids in separating the molecules by size. Typically, after the applied electrical field has been removed, the molecules may be stained. After staining, the separated macromolecules can be seen in a series of bands spread from one end of the electrophoretic medium to the other. If these bands are sufficiently distinct, the molecules in these zones can be examined and studied separately by fixing macromolecules and washing the electrophoretic medium to remove non-fixed components and remaining buffer solution.

Separating lipoprotein particles in bodily fluids (e.g., serum or plasma) provides information on the levels of various lipoprotein particles. Various disease states are linked to levels of apolipoproteins and/or lipoprotein particles including, but not limited to, cardiovascular disease, Alzheimer's disease, hyperlipidemia, abetalipoproteinemia, hypothyroidism, liver disease, diabetes mellitus, and renal problems. Accurate predictors of the risk of an individual of developing various diseases related to lipoprotein particles are needed for research, diagnostic, and therapeutic purposes.

Advances in understanding of the physiological nature of individual lipoprotein types and the effects on human health make it imperative to understand populations of lipoprotein particles and subforms, each of which is the result of, and participates in, specific metabolic processes. Such processes may be good or bad for a particular patient's health, having consequences for therapeutic efforts, including pharmacological therapy, lifestyle changes, diet changes, or other medical intervention.

Apo(a) is one such protein that partly comprises the Lp(a) particle. Although there have been recent improvements in quantifying particle numbers of the various lipoprotein particles, particularly Lp(a) (see e.g., Marcovina et al., “Effect on the Number of Apolipoprotein(a) Kringle 4 Domains on Immunochemical Measurements of Lipoprotein(a),” Clin. Chem. 41 (2): 246-255 (1995); Marcovina et al., “Identification of 34 Apolipoprotein(a) Subforms: Differential Expression of Apolipoprotein(a) Alleles Between American Blacks and Whites,” Biochem Biophys Res Commun 191:1192-6 (1993); Lackner et al., “Molecular Basis of Apolipoprotein(a) Subform Size Heterogeneity as Revealed by Pulsed-Field Gel Electrophoresis,” J Clin Invest 87:2153-61 (1991); Kraft et al., “Apolipoprotein(a) Kringle IV Repeat Number Predicts Risk for Coronary Heart Disease,” Arterioscler Thromb Vasc Biol. 16 (6):713-9 (1996), all of which are hereby incorporated by reference in their entirety), further improvements are needed to permit efficient and cost-effective identification of Lp(a) subforms.

The present invention is directed to overcoming these and other deficiencies in the art.

### SUMMARY

According to aspects illustrated herein, there is provided a method for determining the composition of individual Lp(a) subforms in a test sample. This method involves: (a) providing a test sample comprising Lp(a) subforms obtained from a subject; (b) separating the Lp(a) subforms in the test sample along an electrophoretic gel; (c) measuring the migration velocity of the individual Lp(a) subforms along the electrophoretic gel; (d) comparing, based on the measuring, the migration velocity of the individual Lp(a) subforms to a reference value; and (e) determining, based on the comparing, the molar mass of the individual Lp(a) subforms.

According to aspects illustrated herein, there is provided a method for predicting cardiovascular health. The method involves obtaining a sample from a patient; measuring the size distribution of Lp(a) subforms in the sample; characterizing the patient's risk of cardiovascular disease based on Lp(a) subform sizes and/or distribution.

According to aspects illustrated herein, there is provided a method for predicting cardiovascular health. The method involves obtaining a sample comprising Lp(a) subforms from a patient; separating the Lp(a) subforms in the test sample along an electrophoretic gel; measuring particle number of individual Lp(a) subforms in the sample; and determining a cardiovascular risk value for the subject based on the measured particle number of the Lp(a) subforms.

As noted above, although there have been recent improvements in quantifying particle numbers of the various lipoprotein particles, particularly Lp(a), further improvements are needed. The method described herein significantly improves lipoprotein immuno-fixation electrophoresis (“Lipo-IFE”) methods and devices capable of separating and quantifying particle numbers of the various lipoprotein particles. This technology offers a method for the efficient and cost-effective measurement of specific lipoparticles, rather than mixtures of lipoproteins or separated proteins that do not preserve the information about lipoprotein populations in patient tissues.

For instance, as set forth in the Examples, infra, concerted lipoprotein immuno-fixation electrophoresis protocols with a non-denaturing zonal gel capable of isolating Lp(a)-particles (“Lp(a)-P”) were carried out where Lp(a)-P migration velocities are proportional to apo(a) molecular weights. Prior to these studies, no method existed to characterize the molecular weights of apo(a) subforms in a non-denaturing manner, which preserves information about the apolipoprotein-lipid particle combinations as complete lipoproteins. Such a failure results in a need for inefficient multiple analysis steps or insufficient characterization of a patient's lipoprotein profile. In one embodiment illustrated herein, a simple rapid single zonal gel IFE method is described that is suitable for population screening to provide both Lp(a) particle number and apo(a) subform size. Methods described herein have the advantage of not only a short time to analyze a sample (approximately 90 minutes), but they are also cost-effective. Such a method is a significant improvement on existing technology.

### BRIEF DESCRIPTION OF THE DRAWINGS

FIGS. 1A and 1B show variable Lp(a)-P migration rates via the results of a Lipo-IFE protocol, as described herein. FIG. 1A provides a basic example gel for evaluating differences in Lp(a) migration. FIG. 1A labels electrophoretic banding on the example gel corresponding to LDL, vLDL, median Lp(a), slower cathodic Lp(a), and faster anodic Lp(a). In FIG. 1B, the results of 5 samples run according to the protocols in this application are shown. Samples all contain LDL and VLDL, and all but the negative control further includes Lp(a) particles. The reference (known) Lp(a) content comprises an apo(a) moiety of 600-650 kD. An “anodal” sample in lane 2 contains Lp(a) with an apo(a) moiety of greater than 650 kD, the “mid” sample in lane 3 contains apo(a) moieties of the same mass as the reference, 600-650kD, and the “cathodal” sample contains apo(a) moieties of less than about 600 kD. FIGS. 1A and 1B illustrate the principle of differential detection and quantification of Lp(a) subforms.

FIG. 2 shows examples of samples with differential Lp(a)-P migration bias on zonal gels, separated via the Lipo-IFE protocol described herein. In FIG. 2, a series of samples have been run on a gel in parallel on the Lipo-IFE system. The cathode and anode ends of the gel are labeled and solid lines represent the position of Lp(a) particles after separation where apo(a) mass is about 600-650 kD, that distinguishes large (anodal) from small (cathodal) particles. Four samples have been highlighted (sample numbers 10, 73, 24, and 44). These samples show distinct Lp(a) subform size difference due to the migration rates of smaller subforms (samples 10 and 73, with dashed outline and positioned toward the cathodal end of the substrate) and larger subforms (samples 24 and 44, with a solid outline and positioned toward the anodal end of the substrate). “DBL” indicates the presence of two apo(a) isoforms in a lane.

FIG. 3 compares the zonal gel (Lipo-IFE protocol, inset) shown in FIG. 2 to the same samples in a western blot. In FIG. 3, samples 24, 73, 44, and 10 are further analyzed in a western blot analysis after apo(a) removal from the Lp(a) particles. Western Blot analysis was carried out using standard protocols with Apolipoprotein(a) Isoform Analysis (AAISO), using the Novex WesternBreeze Chromogenic Western Blot Immunodetection Kit (Invitrogen Life Technologies). Multiple reference standards intersperse the variable lanes. Sample 24, which is an anodal (or larger) Lp(a) particle, exhibits a larger separated apo(a) in the western blot, appearing at around 700 kD and greater than 700 kD. Sample 73, which is a cathodal, (or a smaller particle in the zonal gel) corresponds to a smaller apo(a) moiety around 600 kD in the western blot. Samples 44 and 10 repeat the pattern with an anodal Lp(a) similarly having larger apo(a) bands at ˜700 kD and a cathodal Lp(a) having a smaller apo(a) band at less than 450 kD on the western blot. “DBL” indicates the presence of two apo(a) isoforms in a lane.

FIGS. 4A and 4B show more examples of the Lp(a)-P migration differentials in a high-throughput run (see Example 1 and Guadagno et al., “Validation of a Lipoprotein(a) Particle Concentration Assay by Quantitative Lipoprotein Immunofixation Electrophoresis,” Clin Chim Acta 439:219-224 (2014), incorporated herein by reference in its entirety). High-throughput involves the parallel analysis of a multiplicity of samples on automated instrumentation, in this case the Lipo-IFE (Helena Laboratories). In this experiment, 131 samples were tested, including 2 mouse variants. Samples were chosen based upon their electrophoretic migration velocity (to confirm migration velocity and apo(a) size relationship) and Lp(a)-P to mass indices. In FIGS. 4A and 4B, zonal gels run in parallel show variation among patient samples in a high-throughput experiment. Anodal particles of more than about 650 kD and cathodal particles of less than about 600 kD are identified. Anodal samples include sample numbers 1147, 1200, 0481, 1621, 2420, 2495, and 2618. Cathodal samples include sample numbers 1101 and 2611.

FIG. 5 shows comparison of apo(a) content and apoB content in a series of samples on a zonal gel. In FIG. 5, five samples were probed by anti-apoB sera and anti-apoA sera, showing doublet banding, a product of some subjects having two sizes of apo(a) moieties on their Lp(a) particles. Lp(a) doublet banding is seen on samples 1, 3, 4 & 5; these samples contain both apoB & apo(a) probe response without non-specific protein residue in saline lane. Doublet banding is not artifactual and confirms the sensitivity and resolving power of the present methods. Lp(a) doublet banding can be equal, primarily cathodal, or anodal with differential resolution. Sample 2 has single Lp(a), but contains both apoB and apo(a) response, cathodal to LDL and without non-specific protein residue in the saline lane. The frequency of doublet banding is <1%. Saline probe is used to establish presence of artifactual banding.

FIGS. 6A-6R present data comparing Lp(a)-P zonal migration velocities (inset, from FIGS. 2, 4A, and 4B) associated with increasing MW of apo(a), measured by western blot. In FIGS. 6A-6R, more than 100 samples with Lp(a) zonal migration biases were compared to apo(a) isoform size analysis by western blot analysis, as described above. The results from 19 experiments reflecting the same setup and analysis as shown in FIG. 3 are presented. The results show consistent agreement of the new zonal gel method for analyzing Lp(a)-P subform size with the more intensive analysis of separated apo(a) moieties from the same particles. “DBL” indicates the presence of two apo(a) isoforms in a lane.

FIG. 7 shows fluorescent imaging for labeled anti-apoB antibodies bound to Lp(a), LDL, and VLDL particles. The image is a 3Blot/TBS-Image (wet) (as also described in Guadagno et al., “Validation of a Lipoprotein(a) Particle Concentration Assay by Quantitative Lipoprotein Immunofixation Electrophoresis,” Clin Chim Acta 439:219-224 (2014), incorporated herein by reference in its entirety). In general, following electrophoresis, the gel blocks were removed and a rigid antisera template was placed on the gel. The antibody was diluted 1:4 with normal saline and administered through the template onto the gel for 2 min. Excess antibody was removed by blotting and pressing. Residual matrix antibody was removed by rehydration of the gel in a tris-buffered saline bath for 1 min. These steps were performed three times. The gel was subsequently dried at 56° for 8 min, then stained with Acid Violet and scanned. The top row shows sample 3022 at the dilution levels of 5:1 (A1), 7.5:1 (A2), and 10:1 (A3) Alexa Fluor® fluorescent dye:polyclonal antibody, in respective columns. The same experiment is shown for sample 3052 in the bottom row. There are no observable differences in optical properties between the dilution ratios.

FIGS. 8A-8E show results of an initial comparison of acid violet staining detection methods (A/V) and apoB* fluorescently-tagged antibody labeling methods (wherein anti-apoB antibodies are labeled with a fluorescein derivative such as Alexa Fluor® 488 or similar and incubated with the lipoproteins before washing and analyzed), for Lp(a)-P distinction from LDL-P in a single sample. FIG. 8A shows a native zonal gel separation of sample numbers 3022 and 3052, in respective columns containing duplicate runs, labeled by acid violet staining There are 4 total sample runs, where the LDL is evident as a strong band and Lp(a) as a weak band below it. FIG. 8B shows optical imaging with fluorescence of the same samples, where 3022 is on the top row and 3052 is on the bottom row. The columns correspond to different fluorescent dye:anti-apoB ratios in reagents A1, A2, and A3, previously described. FIG. 8C presents numerical results of the optical density readings of each sample, where %Lpa means percent Lp(a) (lipoprotein a) of total detected sample and %LDL means percent LDL (low-density lipoprotein) of the total detected sample. FIG. 8D is the optical density reading of the acid violet staining of sample 3022 in profile and FIG. 8E is the optical density reading of the fluorescent conjugate of sample 3022 in profile (in the reverse direction of the A/V profile).

FIGS. 9A-9E show results of an initial comparison of acid violet staining detection methods (A/V) and apoB* fluorescently-tagged antibody labeling methods, for Lp(a)-P distinction from LDL and VLDL in a single sample. FIG. 9A shows a native zonal gel separation of samples 3034 and 3051, in respective columns containing duplicate runs, labeled by acid violet staining. There are 4 total sample runs, where the LDL is evident as a strong band and Lp(a) as a weak band below it. FIG. 9B shows optical imaging with fluorescence of the same samples, where 3034 is on the top row and 3051 is on the bottom row. The columns correspond to different fluorescent dye:anti-apoB ratios in reagents A1, A2, and A3, previously described. FIG. 9C presents numerical results of the optical density readings of each sample, where %Lpa means percent Lp(a) (lipoprotein a) of total detected sample and %LDL means percent LDL (low-density lipoprotein) of the total detected sample. Further detailed analysis show the levels of Lp(a), VLDL, and LDL below the Lp(a)/LDL levels. FIG. 9D is the optical density reading of the acid violet staining of sample 3051 in profile and FIG. 9E is the optical density reading of the fluorescent conjugate of sample 3051 in profile (in the reverse direction of the A/V profile).

FIGS. 10A-10E show results from a sample comparing the distinction in anodal (large) Lp(a) subforms and cathodal (small) Lp(a) subforms with acid violet staining detection methods (A/V) and apoB*-fluorescently-tagged antibody labeling methods for Lp(a)-P distinction from LDL and VLDL in a single sample. FIG. 10A shows a native zonal gel separation of sample 0816, in the left column, labeled by acid violet staining. On the left, anti-apoB antibodies are used for labeling, which are found on all of Lp(a), LDL, and VLDL. On the right, anti-apo(a) antibodies are used, which only label the Lp(a) particles. FIG. 10B shows optical imaging with fluorescence of the same sample where labeled. The columns correspond to different fluorescent dye:anti-apoB ratios in reagents A1, A2, and A3, previously described. FIG. 10C presents numerical results of the optical density readings of each sample, where %Lpa means percent Lp(a) (lipoprotein a) of total detected sample and %LDL means percent LDL (low-density lipoprotein) of the total detected sample. Further detailed analysis show the levels of Lp(a), VLDL, and LDL below the Lp(a)/LDL levels. FIG. 10D is the optical density reading of the acid violet staining of sample 0816 in profile and FIG. 10E is the optical density reading of the fluorescent conjugate of sample 0816 in profile (in the reverse direction of the A/V profile).

FIGS. 11A-11E show results from a sample comparing the distinction in anodal (large) Lp(a) subforms and cathodal (small) Lp(a) subforms with acid violet staining detection methods (A/V) and apoB* fluorescently-tagged labeling methods, for Lp(a)-P distinction from LDL and VLDL in a single sample. FIG. 11A shows a native zonal gel separation of sample 2377, in the left column, labeled by acid violet staining On the left, anti-apoB antibodies are used for labeling, which are found on all of Lp(a), LDL, and VLDL. On the right, anti-apo(a) antibodies are used, which only label the Lp(a) particles. FIG. 11B shows optical imaging with fluorescence of the same sample where labeled. The columns correspond to different fluorescent dye:anti-apoB ratios in reagents A1, A2, and A3, previously described. FIG. 11C presents numerical results of the optical density readings of each sample, where %Lpa means percent Lp(a) (lipoprotein a) of total detected sample and %LDL means percent LDL (low-density lipoprotein) of the total detected sample. Further detailed analysis show the levels of Lp(a), VLDL, and LDL below the Lp(a)/LDL levels. FIG. 11D is the optical density reading of the acid violet staining of sample 2377 in profile and FIG. 11E is the optical density reading of the fluorescent conjugate of sample 2377 in profile (shown in reverse sequence from A/V).

FIG. 12A-12E show results from a sample comparing the distinction in anodal (large) Lp(a) subforms and cathodal (small) Lp(a) subforms with acid violet staining detection methods (A/V) and apoB* fluorescently-tagged antibody labeling methods, for Lp(a)-P distinction from LDL and VLDL in a single sample. FIG. 12A shows a native zonal gel separation of sample 3389, in the left column, labeled by acid violet staining. On the left, anti-apoB antibodies are used for labeling, which are found on all of Lp(a), LDL, and VLDL. On the right, anti-apo(a) antibodies are used, which only label the Lp(a) particles. FIG. 12B shows optical imaging with fluorescence of the same sample where labeled. The columns correspond to different fluorescent dye:anti-apoB ratios in reagents A1, A2, and A3, previously described. FIG. 12C presents numerical results of the optical density readings of each sample, where %Lpa means percent Lp(a) (lipoprotein a) of total detected sample and %LDL means percent LDL (low-density lipoprotein) of the total detected sample. Further detailed analysis show the levels of Lp(a), VLDL, and LDL below the Lp(a)/LDL levels. FIG. 12D is the optical density reading of the acid violet staining of sample 3309 in profile and FIG. 12E is the optical density reading of the fluorescent conjugate of sample 3389 in profile (shown in reverse sequence from A/V).

FIGS. 13A-13C shows a summary of FIGS. 10-12, comparing doublet-containing samples 0816, 3389, and 2377 to each other on adjacent gels. FIG. 13A shows the zonal gels labeled with acid violet stain for each sample, with apoB labeled in the first column and apo(a) labeled in the second column. FIG. 13B shows the fluorescence detection for each sample, with the samples clearly labeled. FIG. 13C presents the table summarizing each sample and its relative proportion of anodal (large) Lp(a) subform and cathodal (small) Lp(a) subform along with the LDL and VLDL portions of the sample. The methods show good agreement in calculated ratios of each lipoprotein type level independent of subform type and/or lipoprotein type or level.

### DETAILED DESCRIPTION

According to aspects illustrated herein, there is provided a method for determining the composition of individual Lp(a) subforms in a test sample. This method involves: (a) providing a test sample comprising Lp(a) subforms obtained from a subject; (b) separating the Lp(a) subforms in the test sample along an electrophoretic gel; (c) measuring the migration velocity of the individual Lp(a) subforms along the electrophoretic gel; (d) comparing, based on the measuring, the migration velocity of the individual Lp(a) subforms to a reference value; and (e) determining, based on the comparing, the molar mass of the individual Lp(a) subforms.

As noted above, apo(a) is one such protein that partly comprises the Lp(a) particle. Apo(a) may comprise a range of sizes due to the repeats of a particular sequence of amino acids in the protein, a region described as having kringle repeats. See Lackner et al., “Molecular Basis of Apolipoprotein(a) Subform Size Heterogeneity as Revealed by Pulsed-Field Gel Electrophoresis,” J Clin Invest 87:2153-61 (1991); Lackner et al., “Molecular Definition of The Extreme Size Polymorphism in Apolipoprotein(a),” Hum Mol Genet 2:933-940 (1993), each of which is hereby incorporated by reference in its entirety. The number of kringle repeats in apo(a) may range from 10 to greater than 50 repeats. See id. The various sizes of apo(a) due to kringle repeats are called apo(a) subforms or isoforms. Lp(a) is known to be a risk factor for cardiovascular disease and an increase in the number of kringle repeats is inversely correlated with Lp(a) concentration in most, but not all cases. Correspondingly, the size of Lp(a) particles in the blood may have significant importance for cardiovascular health. See Rifai et al., “Apolipoprotein(a) Size and Lipoprotein(a) Concentration and Future Risk of Angina Pectoris with Evidence of Severe Coronary Atherosclerosis in Men: The Physicians' Health Study,” Clinical Chem. 58 (8):1364-1371 (2004); Erqou et al., “Apolipoprotein(a) Isoforms and the Risk of Vascular Disease,” J. Am. Coll. Cardiology 55 (19): 2160-7 (2010); and Thomas Dayspring “Lipoprotein(a),” available at lipidcenter.com/pdfiEntire_Lpa_Complexities (2010), each of which is hereby incorporated by reference in its entirety. Accordingly, determining cardiovascular risk according to aspects described herein may involve assigning the subject to one of a low, moderate, or high cardiovascular risk category. There are well established recommendations for cut-off values for biochemical markers for determining risk (see Rifai et al., “Apolipoprotein(a) Size and Lipoprotein(a) Concentration and Future Risk of Angina Pectoris with Evidence of Severe Coronary Atherosclerosis in Men: The Physicians' Health Study,” Clin. Chem. 58 (8):1364-1371 (2004); Erqou et al., “Apolipoprotein(a) Isoforms and the Risk of Vascular Disease,” J. Am. Coll. Cardiology 55 (19): 2160-7 (2010); and Thomas Dayspring “Lipoprotein(a),” available at lipidcenter.com/pdfiEntire_Lpa_Complexities (2010); BRAUNWALD'S HEART DISEASE: A TEXTBOOK OF CARDIOVASCULAR MEDICINE 9th ed. (Bonow et al. eds. 2011); “Executive Summary of The Third Report of The National Cholesterol Education Program (NCEP) Expert Panel on Detection, Evaluation, And Treatment of High Blood Cholesterol In Adults (Adult Treatment Panel III),” JAMA 285:2486-2497 (2001); “Adult Treatment Panel III (ATP III) of the National Cholesterol Education Program. Implications of Recent Clinical Trials for the National Cholesterol Education Program Adult Treatment Panel III Guidelines,” Circulation 110 (2):227-39 (2004); and MedlinePlus, A service of the U.S. National Library of Medicine and National Institutes of Health available at nlm.nih.gov/medlineplus, each of which is hereby incorporated by reference in its entirety.)

For example, a risk assessment based solely on Lp(a) particle number may be modified by the predominance of large or small isoforms. In this way the isoform size can be used as a weighting factor among a plurality of additional markers. Alternatively, the isoform size may be evaluated independently of the additional markers. In one embodiment, predominantly small isoforms (e.g., less than about 640 kD) may be assigned to a high-risk category, while predominantly large isoforms (e.g., more than about 640 kD) may be assigned to low-risk category. The isoform cutoff between small and large distinctions may vary in the range of 600 kD to 700 kD. The difference between small and large isoform size may be characterized at, for example, about 600 kD, 605 kD, 610 kD, 615 kD, 620 kD, 625 kD, 630 kD, 635 kD, 640 kD, 645 kD, 650 kD, 655 kD, 660 kD, 665 kD, 670 kD, 675 kD, 680 kD, 685 kD, 690 kD, 695 kD, or 700 kD. In one embodiment, high-, moderate-, and low-risk particle sizes may be separated into categories of about <600 kD, about 600-640 kD, and about >640 kD, respectively. In one embodiment, high-, moderate-, and low-risk particle sizes may be separated into categories of about <600 kD, about 600-700 kD, and about >700 kD, respectively. A patient's risk may also be characterized by low risk if they have two large-isoform versions of the apo(a), by moderate risk if they have one large and one small isoform, and high risk if they have two small isoforms. Alternatively, a patient may be characterized as low-, moderate-, or high-risk by taking an average weight of the isoforms present in their sample and categorizing the average as low-, moderate-, or high-risk by their location among a population values split into tertiles. Another method for stratification may involve the estimation of kringle IV repeats and stratifying risk according to tertiles of the number of kringle IV repeats on an apo(a). For example, the high-risk category may include apo(a) with less than 19 kringle repeats, moderate-risk with 19-29 kringle repeats and low-risk greater than 30 kringle repeats. Importantly, the risk categories need not be symmetric. In many cases, only the top quintile is considered high-risk, which would involve fewer than 19 kringle repeats, and the moderate and low-risk categories are split among the remaining population distribution. The categories may be determined by a longitudinal study comparing outcomes versus apo(a) sizes among patients in a study.

Apolipoprotein a (“apo(a)”) is highly heritable and mainly controlled by the apo(a) gene [LPA] located on chromosome 6q26-27. Apo(a) is co-dominantly expressed and therefore both should be present and detectable for a patient with genes for two different isoforms. Doublets in the AAISO system and method presented herein confirm its sensitivity and resolving power through the precise control of migration velocity. The method permits population studies to further validate the apo(a) diversity among patients. Further, doublets of significantly different apo(a) size are “mis-determined” by conventional Lp(a)-mass assays, which only provide for an average Lp(a)-mass without regard to divergent phenotype. Given the cardiovascular health risks associated with Lp(a)-P with various apo(a) isoforms, the methods described herein offer a more accurate method for clinical study, clinical risk assessment, and CVD diagnosis, because it can discriminate and quantitate the individual molar contributions of each individual subform.

Herein is described, in one aspect, a method to determine the molecular weight (“MW”) of apo(a) on intact/native Lp(a)-Particles (i.e., non-denatured). As used herein, the term molecular weight may refer to molar mass. Intact or native Lp(a)-Particles include those that have not been subjected to denaturing treatment such as treatment with sodium dodecyl sulfate (“SDS”) delipidation, reduction or removal of the apolipoprotein from the intact particle. Given the atherogenic differences associated with large and small Lp(a), (references in blot versus zonal presentation), an algorithm may be established for CVD risk relative to Lp(a) particle number mitigated by subform size.

Gel electrophoresis is a technique used to separate molecules based on their size and charge, according to the following equation: V=EZ/F, where V=the rate (velocity) of migration, E is the strength of the electrical field, Z is the charge on the molecule and F is the frictional force on the molecule. In zonal gel electrophoresis, cations (positively charged) in solution migrate toward the cathode of gel electrophoresis (negatively charged) whereas anions (negatively charged) migrate toward the anode of gel electrophoresis (positively charged) when an electrical field is applied. The migration velocity is proportional to the ratio between the charges of the protein and its mass. The higher charge per unit of mass, the faster the migration. It is noted that in denaturing gels (as distinguished from zonal gels), proteins are denatured by adding a detergent such as SDS, to separate them exclusively according to molecular weight (Shapiro et al., “Molecular Weight Estimation of Polypeptide Chains by Electrophoresis in SDS-Polyacrylamide Gels,” Biochem. Biophys. Res. Commun. 28: 815-820 (1967), which is hereby incorporated by reference in its entirety). SDS is a mild reducing agent which maintains the polypeptides in a charged denatured state once the protein has been exposed to strong reducing agents to reduce the disulfide bonds to sulfhydryls. As apo(a) is held to apoB by disulfide bonds, such reduction effectively releases apo(a) from the Lp(a) particle. SDS gives the molecule a net negative charge that allows migration through the gel in direct relation to size. In addition, denaturation disrupts secondary, tertiary, and quaternary structure and therefore migration velocity is proportional to size and not to biomolecular structure. Zonal gels are run under non-denaturing conditions that will not alter the structure of the proteins; separation is primarily proportional to the negative charge on the molecule (caused by the gel pH˜9) and only secondarily by size (due to the hydro resistance of the particles within the buffered gel matrix augmented by electroendosmosis). Zonal gels are described in, e.g., Jeppsson et al., “Agarose Gel Electrophoresis,” Clin. Chem. 25/4: 629-638 (1979), which is hereby incorporated by reference in its entirety.

Basically, the larger the apolipoprotein on the lipid particle, the greater will be the overall negative charge, (Z). Frictional forces, (F) on the particles are minimal due to the large gel pore matrix relative to the size of the lipid particle and reduced to subordinate hydrodynamic resistance. Therefore, V≈EZ and at constant voltage, V is proportional to Z. For Lp(a) subform separation, Lp(a)-P is a lipid particle with a single apoB, (MW≈540 kD) and a single apo(a). Subforms of apo(a) however can vary from 300 kD to 900 kD. The overall charge on the Lp(a)-P is the sum of the charges from both apoB and the particular polymorphic isoform of apo(a). As apoB has a constant MW, any variation in charge for the Lp(a)-P will be a function of apo(a) size. Migration velocity (V is proportional to Z) will be a function of the differential charge from the apo(a) subform, which is directly proportional to apo(a) size (i.e., apo(a) MW).

The terms “lipoprotein particle,” “lipid protein particle,” “lipid particle,” and the like as used herein refers to a particle that contains both protein and lipid. Examples of lipoprotein particles are described in more detail below.

The term “lipoprotein particle number” as used herein refers to the molar concentration (nmol/L) of lipoprotein particles present in the bodily fluid. Particle number may be measured as molar concentration in nmol/L.

The term “apolipoprotein” as used herein refers to a protein that combines with lipids to form a lipoprotein particle. Examples of apolipoprotein types are described in more detail below. The unique nature of the apolipoprotein is their stoichiometric relationship to lipoprotein particles, providing an estimate of the lipoprotein particle number, which is described in more detail below.

Suitable biological samples or biosamples according to the invention include human biological matrices, urine, cerebrospinal fluid, whole blood, plasma, serum, and human lipoprotein fractions. For example, the sample may be fresh blood or stored blood or blood fractions. The sample may be a blood sample expressly obtained for the assays of this invention or a blood sample obtained for another purpose which can be subsampled for use in accordance with the methods according to the invention. For instance, the biological sample may be whole blood. Whole blood may be obtained from the subject using standard clinical procedures. The biological sample may also be plasma. Plasma may be obtained from whole blood samples by centrifugation of anti-coagulated blood. The biological sample may also be serum. The sample may be pretreated as necessary by dilution in an appropriate buffer solution, concentrated if desired, or fractionated by any number of methods including but not limited to ultracentrifugation, fractionation by fast performance liquid chromatography (FPLC), or precipitation. Any of a number of standard aqueous buffer solutions, employing one of a variety of buffers, such as phosphate, Tris, or the like, at physiological to alkaline pH can be used.

Methods as described herein may be used with any suitable gel electrophoresis system and/or method. In the description of the physical structure and methods, the Examples and corresponding figures show embodiments of the gels used and results of methods described herein. Suitable gel electrophoresis systems and methods include, for example, those described in WO 2013/181267 and U.S. Patent Application Publication No. 2012/0052594, each of which is hereby incorporated by reference in its entirety. Apparatuses for the detection of Lp(a)-P that may be used in accordance with methods as described herein include those of U.S. Patent Application Publication No. 2012/0052594, which is hereby incorporated by reference in its entirety. In immunofixation methods, such as described in U.S. Patent Application Publication No. 2012/0052594, which is hereby incorporated herein by reference in its entirety, a biological sample (e.g., serum) is applied to a substrate and the components are electrophoresed. Anti-sera containing labeled antibodies (e.g., anti-ApoB antibodies) that target specific components of the blood is applied to the substrate. The antibodies attach to their antigen targets, and the targets can be identified through some means of detecting the label.

As described in more detail below, methods described herein may also employ unlabeled antibodies that are detected by contacting the gel with a protein dye (e.g., acid violet or the like). Accordingly, the methods described herein may involve contacting the electrophoretic gel with a protein dye that dyes apoB on the individual Lp(a) particles and subsequently detecting the dyed Lp(a) subforms. Dye binding is associated with dye bound to anti-apoB to apoB. This represents the primary signal. Dye bound to apo(a) is secondary signal noise relative to the signal produced by the PAb-apoB-A/V. The protein dye may be acid violet. In non-limiting examples, the protein dye may also be a Coomassie dye, a derivative or improvement thereof such as Bio-Safe® (Bio-Rad, Hercules, Calif.) or SimplyBlue® (Invitrogen, Carlsbad, Calif.) or Imperial Protein Stain (Thermo Fisher Scientific, Rockford, Ill.), a silver-based stain, a zinc-based stain, a fluorescent dye, or there may be a functional group specific stain such as glycoprotein stain, phosphoprotein stain, His-tag stain, or any other type or combination of stains. Such methods are well known by those skilled in the art, as described in, for example, H. J. CONN'S BIOLOGICAL STAINS (R. D. Lillie ed., Williams & Wilkins 1977) or in commercial literature from Thermo Scientific (e.g. Pierce Protein Biology Products) incorporated herein by reference. One significant advantage of the methods described herein is that they may be carried out without the use of fluorescence involved with assay techniques commonly used to detect Lp(a)-p. Such techniques not only require expensive reagents, but are time intensive, many requiring multiple days to complete. Thus, methods described herein have the advantage of not only a short time to analyze a sample (approximately 90 minutes), but they are also cost-effective. Accordingly, in certain methods described herein, the methods do not involve use of fluorescence for detection of Lp(a)-p.

As noted above, methods described herein may involve fixing the separated individual Lp(a) subforms within the gel prior to detection. Fixing the separated individual Lp(a) subforms may be carried out by contacting the gel with a suitable antibody (or anti-sera containing antibodies), as is well-known. The antibody may be an anti-ApoB antibody. Such fixation may be followed by contacting the gel with a protein stain (such as acid violet, as described herein) and detection of the stained Lp(a)-P subforms using a densitometer.

The gel electrophoresis may be one-dimensional or two-dimensional. Isoelectric focusing may also be performed. Electrophoretic gel substrates suitable for use with the invention are known to those of skill in the art. For instance, suitable gel substrates include, but are not limited to, agarose or polyacrylamide or blends of the two. As discussed above, SDS-PAGE (polyacrylamide) gels separate proteins based on their size because the SDS coats the proteins with a negative charge. Separation of proteins on the agarose gel is by charge. Accordingly, as also noted above, embodiments described herein may use zonal gel electrophoresis. Zonal gel electrophoresis, wherein non-denatured proteins are separated by charge offers the benefit of a simple high-resolution protocol.

Electrophoretic gels of varying sizes may contain various numbers of lanes and rows (e.g., one, two, three, four, five, six, seven, eight, nine, ten, etc.). The biological sample from a single individual or subject may be probed to identify multiple components and/or serum from multiple individuals may be tested. The protocols for conducting electrophoresis on different sizes of gels will be similar except that modifications may be made to optimize separation on that size of gel.

Methods according to aspects described herein may also include characterizing, based on the determining of the molar mass of the individual Lp(a) subforms, at least two Lp(a) subforms of different molar mass. Such characterizing may include determining the level or concentration of the individual Lp(a) subforms having a molar mass to be within a certain range (e.g., less than about 600 kD, between about 600 kD and 700 kD, or greater than about 700 kD). Determining the concentration or level of the particular Lp(a) subform in the sample may involve densitometric characterization of the Lp(a) subforms that are separated along the electrophoretic gel. Use of densitometry to detect and determine, e.g., concentration of a detected protein is described in more detail below. The method may further involve determining, based on the characterization of at least two Lp(a) subforms of different molar mass, an Lp(a) subform size distribution of the characterized Lp(a) subforms.

Methods according to aspects described herein may also include measuring the total particle number of the Lp(a) present in the sample, the individual Lp(a) subforms, or both.

By way of background, electrophoresis measures relative concentrations, i.e., percentage fractions are calculated as the area under curves from detected bands that have been translated into signals to produce electropherograms. In particular, after electrophoresis, a gel may be stained with a protein dye (e.g., Acid Violet) and passed through the optical system of a densitometer to create an electrophoregram, a visual diagram or graph of the separated bands. A densitometer is a special spectrophotometer that measures light transmitted through a solid sample such as a stained gel. Absorbance can be measured with densitometry and fluorescence can be measured with photon-counting imaging protocols such as provided by BioRad Chemidoc® instruments. Using the optical density measurements, the densitometer represents the detected bands of stain as peaks. These peaks compose the graph or electrophoregram and are printed on a recorder chart or computer display. An integrator or microprocessor evaluates the area under each peak and reports each as a percent of the total sample. For example, if the electrophoresis is being used for separation of serum proteins, the concentration of each band is derived from this percent and the total protein concentration.

Because Lp(a) subforms have a known stoichiometric relationship with Apo B (i.e., 1:1 stoichiometry of Apo B:Lp(a)-P), the particle number of the individual Lp(a) subforms may also be determined by measuring the apoB concentration of the particular subform separated along the electrophoretic gel. The particle number may be quantified by comparison with a separate analysis that characterizes the total lipid particle or class of lipid particle concentration in the sample. Such separate analysis may be ultracentrifugation (UC), NMR, or any other analysis method that can characterize a concentration or total particle number for particles in the sample. However, neither UC or NMR can estimate Lp(a)-P directly or independently. Both can only estimate the sum total of LDL-P and Lp(a)-P. Correlations have been shown between UC, NMR and electrophoresis. Those comparisons use samples with and without Lp(a)-P and only show acceptable correlations between the methods when both Lp(a)-P and LDL-P from electrophoresis are included. The sample used for Lipo-IFE and lipid particle quantification may be different aliquots of the same sample. Densitometer software may also automatically calculate and print the relative percent and the mg/dL for each band along an electrophoretic gel when the specimen total Apo-B is entered as (% of Fraction)×(total Apo-B). The particle number is calculated in a preferred laboratory information system as (mg/dL)/0.054=PN. The conversion factor (mg/dL)/0.054 is calculated from:

[ApoB (mg/dL)×(10 dL/L)×(106 nmol/mmol)]/[Molecular Mass of apoB (540,000 mg/mmol)]

Results are reported as Particle Number (PN=nmol/L).

Separating Lp(a) subforms in the test sample along an electrophoretic gel may include separating the Lp(a) subforms in the test sample along a first lane of the electrophoretic gel. Comparing the migration velocity of the individual Lp(a) subforms to a reference value may involve comparing the migration velocity of the individual Lp(a) subforms to a control sample that is separated along a second lane of the electrophoretic gel.

Conducting electrophoresis may involve carrying out the step of depositing a sample in a receiving well of an electrophoretic gel as part of a method for assessing the level of and/or molecular mass of the Lp(a) subforms present in a bodily fluid, as described in U.S. Patent Application Publication No. 2012/0052594, which is hereby incorporated by reference in its entirety. The exemplary method involves separating lipoprotein particles present in a bodily fluid sample by gel electrophoresis on a gel electrophoresis substrate, exposing the substrate to an antibody to detect an immunologically active agent associated with lipoprotein particles or components of lipoprotein particles, exposing the substrate to a reagent for detection of the presence of proteins or lipids, and determining the level and/or molecular mass of the Lp(a) subforms.

Methods described herein may also be carried out in conjunction with in-situ calibration (as described in U.S. Patent Application Publication No. 2013/0319864, which is hereby incorporated by reference in its entirety) and involve combining a volume of a test sample with a volume or quantity of a calibrating sample to form a final volume, in which the volume or quantity of the calibrating sample includes a known concentration of a calibrator and the final volume includes a known ratio of test sample to calibrating sample. Alternatively, a known mass of dry calibrator may be mixed into the test sample to provide a known concentration of calibrator and no volume change. The method also includes depositing a loading fraction in a receiving well, electrophoretic gel, in which the loading fraction is a fraction of the final volume and separating the loading fraction along a common separation lane of the electrophoretic gel such that components of the test sample and the calibrator are separated from one another along the common separation lane. The method also includes detecting the calibrator and separated components of test sample within the common separation lane and measuring the level of the calibrator and separated components of the test sample based on the detecting, thereby performing electrophoresis with in-situ calibration.

Accordingly, the reference value as described herein may be a control Lp(a) subform separated along the electrophoretic gel. The reference value may also be a predetermined value used for comparison.

The method can distinguish Lp(a) particles with apo(a) proteins of molecular weights, for example, greater than 700 kD, less than 600 kD and between 600 and 700 kD. In one embodiment, the molar mass of apo(a) protein of the individual Lp(a) subforms is greater than 600 kD. In one embodiment, the molar mass of apo(a) protein of the individual Lp(a) subforms is determined to be greater than 700 kD. In one embodiment, the molar mass of apo(a) protein of the individual Lp(a) subforms is between 600 and 700 kD. In one embodiment, the molar mass of apo(a) protein of the individual Lp(a) subforms is less than 600 kD.

Determining the molar mass of the individual Lp(a) subforms according to aspects illustrated herein may involve assigning the individual Lp(a) subforms to one of a low, mid, or high molar mass category. For instance, the individual Lp(a) subforms having a molar mass less than about 600 kD may be assigned to the low molar mass category; individual Lp(a) subforms having a molar mass of between about 600 kD and 700 kD may be assigned to the mid molar mass category; and individual Lp(a) subforms having a molar mass of greater than about 700 kD are assigned to a high molar mass category.

According to aspects illustrated herein, the individual Lp(a) subforms of the test sample may each be bound to a signal-producing molecule capable of producing or causing production of a detectable signal. Accordingly, in one embodiment, the method also involves contacting the separated individual Lp(a) subforms of the test sample bound to the signal-producing molecule with a reagent capable of interacting with the signal-producing molecule, where the signal-producing molecule produces the detectable signal upon contact with the reagent and where said method further comprises detecting the detectable signal.

In one embodiment, the individual Lp(a) subforms of the test sample are bound to signal-producing molecules that are distinguishable from one another.

Suitable systems and methods involving signal-producing molecules that are distinguishable from one another include those for use in in situ detection of lipid particles within an electrophoretic matrix, as describe in U.S. Patent Application Publication No. 2014/0243431, which is hereby incorporated by reference in its entirety.

Such a system includes a gel substrate to receive a biological sample and at least two lipoprotein-binding complexes. Each complex includes an antibody that binds a lipoprotein particle or a portion thereof, where the antibody is bound to a signal-producing molecule capable of producing or causing production of a detectable signal. Each detectable signal of the at least two lipoprotein-binding complexes is distinguishable from the other detectable signal. The system also includes a device for detecting the detectable signal, where the detecting indicates the level of the specific Apolipoproteins and/or lipoprotein particles in the biological sample.

As used herein, the term “antibody” is meant to include intact immunoglobulins derived from natural sources or from recombinant sources, as well as immunoreactive portions (i.e. antigen binding portions) of intact immunoglobulins. The antibodies of the invention may exist in a variety of forms including, for example, polyclonal antibodies, monoclonal antibodies, intracellular antibodies, antibody fragments (e.g., Fv, Fab and F(ab)2), as well as single chain antibodies (scFv), chimeric antibodies and humanized antibodies (Ed Harlow and David Lane, USING ANTIBODIES: A LABORATORY MANUAL (Cold Spring Harbor Laboratory Press, 1999); Houston et al., “Protein Engineering of Antibody Binding Sites: Recovery of Specific Activity in an Anti-Digoxin Single-Chain Fv Analogue Produced in Escherichia coli,” Proc Natl Acad Sci USA 85:5879-5883 (1988); Bird et al, “Single-Chain Antigen-Binding Proteins,” Science 242:423-426 (1988), which are hereby incorporated by reference in their entirety).

Methods for monoclonal antibody production may be carried out using techniques well-known in the art (MONOCLONAL ANTIBODIES—PRODUCTION, ENGINEERING AND CLINICAL APPLICATIONS (Mary A. Ritter and Heather M. Ladyman eds., 1995), which is hereby incorporated by reference in its entirety). Procedures for raising polyclonal antibodies are also well known (Ed Harlow and David Lane, USING ANTIBODIES: A LABORATORY MANUAL (Cold Spring Harbor Laboratory Press, 1988), which is hereby incorporated by reference in its entirety).

For example, polyclonal antibodies may be produced by injecting a suitable animal host, such as a rabbit, with the lipoprotein of interest and an adjuvant. Approximately 0.02 milliliters may be injected, with reinjection occurring every 21 days until peak antibody titer is achieved. Antibody titer may be tested by, for example, an ear bleed. Antibodies to Apo B-100 or other apolipoprotein may be produced in this manner. Alternatively, antibodies to Apo B-100 or other apolipoprotein may be purchased commercially.

Antibodies can be generated with high levels of specificity, sufficient to distinguish different portions of the same proteins, such as different kringles on apo(a), in particular repeating kringle IV and any other kringle on apo(a). As Lp(a) subforms are distinguished by the number of kringle repeats, characterizing kringle content can facilitate identification of Lp(a) size with great detail. Such antibodies would be labeled with, for example, different color fluorescent probes (as described above) and the apolipoprotein type can be distinguished with extreme detail. As described below, absolute levels and ratios of detailed measurements can be reported and converted into a risk factor. For example the ratio of small to large Lp(a) can be reported with a specific cutoffs for high-, medium-, and low-risk ranges.

In addition to whole antibodies, the invention encompasses binding portions of such antibodies. Such binding portions include the monovalent Fab fragments, Fv fragments (e.g., single-chain antibody, scFv), single variable V_{H} and V_{L} domains, and the bivalent F(ab′)₂ fragments, Bis-scFv, diabodies, triabodies, minibodies, etc. These antibody fragments can be made by conventional procedures, such as proteolytic fragmentation procedures, as described in James Goding, MONOCLONAL ANTIBODIES: PRINCIPLES AND PRACTICE 98-118 (Academic Press, 1983) and Ed Harlow and David Lane, ANTIBODIES: A LABORATORY MANUAL (Cold Spring Harbor Laboratory, 1988), which are hereby incorporated by reference in their entirety, or other methods known in the art.

Suitable signal-producing molecules that are capable of producing or causing production of a detectable signal will be known to those of skill in the art. The detectable signal includes any signal suitable for detection and/or measurement by radiometric, colorimetric, fluorometric, size-separation, or precipitation means, or other means known in the art.

Examples of signal-producing molecules that are capable of producing or causing production of a detectable signal include various enzymes, prosthetic groups, fluorescent materials, luminescent materials, bioluminescent materials, radioactive materials, positron emitting metals, and nonradioactive paramagnetic metal ions. The signal-producing molecules may be coupled or conjugated either directly to the antibody or indirectly, through an intermediate (such as, for example, a linker known in the art) using techniques known in the art. See, for example, U.S. Pat. No. 4,741,900 for metal ions which can be conjugated to antibodies for use as diagnostics according to the invention. Further examples include, but not limited to, various enzymes. Examples of enzymes include, but are not limited to, horseradish peroxidase, alkaline phosphatase, beta-galactosidase, or acetylcholinesterase; prosthetic group complexes such as, but not limited to, streptavidin/biotin and avidin/biotin. Examples of fluorescent materials include, but are not limited to, umbelliferone, fluorescein, fluorescein isothiocyanate, rhodamine, dichlorotriazinylamine fluorescein, dansyl chloride or phycoerythrin. Examples of luminescent material include, but are not limited to, luminol. Examples of bioluminescent materials include, but not limited to, luciferase, luciferin, and aequorin. Examples of radioactive material include, but are not limited to, bismuth (213Bi), carbon (14C), chromium (51Cr), (153Gd, 159Gd)5 gallium (68Ga, 67Ga), germanium (68Ge), holmium (166Ho), indium (115In, 113In, 112In, 111In), iodine (1311, 1251, 1231, 1211), lanthanium (140La), lutetium (177Lu), manganese (54Mn), molybdenum (99Mo), palladium (103Pd), phosphorous (32P), praseodymium (142Pr), promethium (149Pm), rhenium (186Re, 188Re), rhodium (105Rh), ruthemium (97Ru), samarium (153Sm), scandium (47Sc), selenium (75Se), strontium (85Sr), sulfur (35S), technetium (99Tc), thallium (201Ti), tin (113Sn, 117Sn), tritium (3H), xenon (133Xe), ytterbium (169Yb, 175Yb), yttrium (90Y), zinc (65Zn). Further examples include positron emitting metals using various positron emission tomographies, and nonradioactive paramagnetic metal ions.

Detection of an antibody-signal producing molecule complex in accordance with the invention may also be achieved by addition of a reagent capable of interacting with the signal-producing molecule, where the signal-producing molecule produces a detectable signal upon contact with the reagent. For example, light is emitted when luciferase acts on the appropriate luciferin substrate.

A secondary antibody that is coupled to a detectable signal or moiety, such as for example, an enzyme (e.g., luciferase), fluorophore, or chromophore may also be used.

As noted above, each detectable signal of the at least two lipoprotein-binding complexes may be distinguishable from the other detectable signal. This permits cocktailing at least two lipoprotein-binding complexes where each of the complexes detects a different Lp(a) subform or a portion thereof, each complex also producing or capable of producing a different detectable signal. For example, a first lipoprotein-binding complex may include fluorescein isothiocyanate (FITC)-labeled antibody which binds a kringle IV repeat on the apo(a) for all Lp(a) components in a sample. A second non-kringle IV-binding complex may include rhodamine-labeled antibody which binds a second portion of apo(a). The first and second complexes may be mixed or cocktailed together. This permits probing of multiple antigens in a single electrophoretic location. The ratios of intensities from the kringle IV repeats to non-kringle IV components of apo(a) will facilitate a more accurate measurement of Lp(a) subform size, when compared to a known kringle IV/non-kringle IV standard.

For example, the signal-producing molecules may include fluorescent tags. Fluorescence tagging and the detection of natural fluorescence in molecules is a method of analytical chemistry and biology that is well known in the art. The instruments used to detect fluorescence may include the following components. A light source with a broad optical bandwidth such as a light bulb or a laser is used as the source of the stimulating light. An optical filter is used to select the light at the desired stimulation wavelength and beam it onto the sample. Optical filters are available at essentially any wavelength and are typically constructed by the deposition of layers of thin film at a fraction of the wavelength of the desired transmission wavelength. The light that exits the optical filter is then applied to the sample to stimulate the fluorescent molecule.

The molecule then emits light at its characteristic fluorescent wavelength. This light is collected by a suitable lens and is then passed through a second optical filter centered at the characteristic wavelength before being brought to a detection device such as a photomultiplier tube, a photoconductive cell, or a semiconductor optical detector. Therefore, only light at the desired characteristic wavelength is detected to determine the presence of the fluorescent molecule. Accordingly, the at least two lipoprotein-binding complexes may include fluorescent molecules that emit light at different, distinguishable fluorescent wavelengths.

Fluorescent tags may be multiplexed in a single area such that they are optically distinct. For example, 5 different fluorescent tags, red, green, blue, yellow, and orange may be applied to the same limited area and be independently detected and distinguished by optical detection software. For example, the Life Technologies Alexa Fluor product line includes at least 19 distinct dyes that may be combined for tagging distinct antibodies to label and identify individual antigens. For example, as shown in the Examples described herein, Alexa 647, Alexa 546 and Alexa 488 may be combined for tagging distinct antibodies to label and identify individual antigens (e.g., Apo B, Apo C-III, and Apo E). Additional fluorophores such as Alexa 430 may be included to optimize a method and avoid cross-talk between labels. An optical system can quantitate the fluorescent signals and automatically normalize the signal value to generate relative densities or particle numbers. For example, by normalizing the extinction/emission coefficients or quantum relativity of each dye, relative values for concentration or particle number can be determined.

The system and methods may also include a device or use of a device for detecting the detectable signal, where the detecting indicates the level of the specific Lp(a) particles in the biological sample. The device may also quantitate the level of specific Lp(a) particles based on the detection of the signal-producing molecule.

The presence of the detected particle or a portion thereof in the electrophoretic gel may then be quantified by measurement of the detectable signal or moiety. The particle number may then be calculated according to known stoichiometric relationships. The particle number may be quantified by comparison with a separate analysis that characterizes the total lipid particle or class of lipid particle concentration in the sample. Such separate analysis may be ultracentrifugation, NMR, or any other analysis method that can characterize a concentration or total particle number for particles in the sample. Said sample used in lipid particle electrophoresis and lipid particle quantification may be different aliquots of the same sample.

A suitable method for such in situ detection include a method of assessing the level of specific Lp(a) subform particles present in a biological sample. This method includes the steps of providing a biological sample containing Lp(a) subform particles and providing at least two lipoprotein-binding complexes. Each complex includes an antibody that binds a Lp(a) subform particle or a portion thereof, where the antibody is bound to a signal-producing molecule capable of producing or causing production of a detectable signal. Each detectable signal of the at least two lipoprotein-binding complexes is distinguishable from the other detectable signal. This method also includes contacting the biological sample with the antibody under conditions suitable to form a lipoprotein-antibody-signal producing molecule complex and separating the lipoprotein particles present in the biological sample by depositing the biological sample on an electrophoretic gel and carrying out gel electrophoresis. This method further includes detecting the detectable signal produced by the signal-producing molecule of the lipoprotein-antibody-signal producing molecule complex on the electrophoretic gel and determining the level of the specific Lp(a) subform particle present in the biological sample based on the detecting.

A further method for in situ detection includes a method of determining whether a subject is at increased risk for cardiovascular disease. This method includes assessing the level of specific Lp(a) subform particles present in a biological sample. The assessing includes the steps of providing a biological sample containing Lp(a) subform particles and providing at least two lipoprotein-binding complexes. Each complex includes an antibody that binds an Lp(a) subform or a portion thereof, where the antibody is bound to a signal-producing molecule capable of producing or causing production of a detectable signal. Each detectable signal of the at least two lipoprotein-binding complexes is distinguishable from the other detectable signal. The assessing step also includes separating the lipid protein particles present in the biological sample by depositing the biological sample on an electrophoretic gel and carrying out gel electrophoresis; contacting the biological sample with the at least two lipoprotein-binding complexes under conditions suitable to form a lipoprotein-antibody-signal producing molecule complex; detecting the detectable signal produced by the signal-producing molecule of the lipoprotein-antibody-signal producing molecule complex on the electrophoretic gel; and determining the level of the specific Lp(a) subform particle present in the biological sample based on the detecting. The method also includes the step of correlating the determined level of the Lp(a) subform particle to a control or reference value to determine if the subject is at an increased risk for cardiovascular disease.

Methods according to aspects described herein may also include measuring the particle number of the individual Lp(a) subforms; determining the size distribution of the Lp(a) subforms in the sample based on the determined molar mass and particle number of the individual Lp(a) subforms; and determining a cardiovascular risk value for the subject based on the determined size distribution of the Lp(a) subforms. Methods according to aspects described herein may also include measuring the particle number of the Lp(a) present in the sample and determining a cardiovascular risk value for the subject based on the measured particular number.

According to aspects illustrated herein, there is provided a method for predicting cardiovascular health. The method involves obtaining a sample comprising Lp(a) subforms from a patient; separating the Lp(a) subforms in the test sample along an electrophoretic gel; measuring particle number of individual Lp(a) subforms in the sample; and determining a cardiovascular risk value for the subject based on the measured particle number of the Lp(a) subforms.

The cardiovascular risk may be determined as, for instance, low (or optimal), moderate, or high, as described above.

Methods described herein may also be carried out where the subject has, or is undergoing, an existing therapeutic regimen. Such methods may involve modifying the therapeutic regimen based on the determined size distribution of Lp(a) subforms, the particle number of the Lp(a), or both. The therapy regimen may also be modified based on a determined cardiovascular risk value, as described above. For instance, the dosage of a statin (or other drug, as described herein) may be increased if the cardiovascular risk value is intermediate or high.

The invention also includes selecting a therapy regimen based on the risk for cardiovascular disease determined. For instance, an individual may be determined to be at an elevated risk according to the methods and a treatment regimen may then be selected based on the elevated risk.

The selected therapy regimen may include administering drugs or supplements. Suitable drugs or supplements include those administered for the purpose of lowering serum cholesterol, lowering LDL, IDL, and VLDL, Lp(a) and/or raising HDL, as known in the art.

Examples of suitable drugs include niacin, an anti-inflammatory agent, an antithrombotic agent, an anti-platelet agent, a fibrinolytic agent, a lipid reducing agent, a direct thrombin inhibitor, a glycoprotein IIb/IIIa receptor inhibitor, an agent that binds to cellular adhesion molecules and inhibits the ability of white blood cells to attach to such molecules, a calcium channel blocker, a beta-adrenergic receptor blocker, an angiotensin system inhibitor, and combinations thereof

In one embodiment, the selected therapy regimen comprises administering a drug selected from the group consisting of niacin, fenofibrate, estrogen, and raloxifene. In one embodiment, the selected therapy regimen includes niacin. In one embodiment, the selected therapy regimen includes a statin. In one embodiment, the selected therapy regimen includes administering niacin and a statin. In one embodiment, the selected therapy regimen includes administering a statin and ezetimibe. In one embodiment, the selected therapy regimen includes administering niacin, ezetimibe, a statin, or a combination thereof

The selected therapy regimen may also involve giving recommendations on making or maintaining lifestyle choices based on the risk for cardiovascular disease determined Lifestyle choices may involve changes in diet, changes in exercise, reducing or eliminating smoking, or a combination thereof

A report may also be generated that includes, among other things, a description of the selected treatment regimen. In some embodiments, the results of lipoprotein analyses are reported in such a report. A report refers in the context of lipoprotein and other lipid analyses to a report provided, for example to a patient, a clinician, other health care provider, epidemiologist, and the like, which includes the results of analysis of a biological specimen, for example a plasma specimen, from an individual. Reports can be presented in printed or electronic form, or in any form convenient for analysis, review and/or archiving of the data therein, as known in the art. A report may include identifying information about the individual subject of the report, including without limitation name, address, gender, identification information (e.g., social security number, insurance numbers), and the like. A report may include biochemical characterization of the lipids in the sample in addition to Lp(a), for example without limitation triglycerides, total cholesterol, LDL cholesterol, and/or HDL cholesterol, and the like. A report may further include characterization of lipoproteins, and reference ranges therefore, conducted on samples prepared by the methods provided herein. The term “reference range” and like terms refer to concentrations of components of biological samples known in the art to reflect typical normal observed ranges in a population of individuals. Exemplary characterization of lipoproteins in an analysis report may include the concentration and reference range for VLDL, IDL, Lp(a), LDL and HDL, and subclasses thereof A report may further include lipoprotein size distribution trends.

The invention also may further include administering the selected treatment regimen to the subject. Accordingly, a further aspect of the present invention relates to a method of treating a subject having an elevated risk for cardiovascular disease determined according to methods described herein.

The invention also relates to a method of monitoring the risk for developing cardiovascular disease. This method includes determining whether a subject is at increased risk for cardiovascular disease at a first time point and repeating the determining at one or more later time points (e.g., before and after therapeutic intervention or at progressive time points during a course of therapeutic intervention). The determined risk at each progressive time point is compared the determined risk from one or more earlier time points to evaluate whether the subject's risk for developing cardiovascular disease has increased or decreased, thereby monitoring the risk for developing cardiovascular disease. This method may involve assigning a risk category based on the determined risk for developing cardiovascular disease and comparing the risk categories assigned at progressive time points (e.g., comparing a first risk category determined at a first time point to a second risk category taken at a second time point), thereby monitoring the risk for developing cardiovascular disease.

According to aspects illustrated herein, there is provided a method predicting cardiovascular health. The method involves obtaining a sample from a patient; measuring the size distribution of Lp(a) subforms in the sample; characterizing the patient's risk of cardiovascular disease based on Lp(a) subform sizes and/or distribution. In one embodiment, a therapeutic regimen is prescribed to the patient to reduce the risk of cardiovascular disease.

### EXAMPLES

The following examples are provided to illustrate embodiments of subject matter claimed herein, but are by no means intended to limit its scope.

### Example 1

### Lp(a) Subform Analysis: Materials and Methods

The following protocols were employed in performing the experiments described herein. Blood samples were acquired from preferably fasting patients. At least 125 μL of serum was prepared from each of the blood samples using well-known methods in the art. Each sample was held at 2-8° C. and tested within 4 days of collection.

A SPIFE 3000 Gel Electrophoresis and Processing Instrument (Helena Laboratories) was prepared by modifying the cholesterol electrophoresis program and IFE stainer with additional drying, blotting, and rehydration protocols according to instructions provided in the Operator's Manual. Prior to operation, the following solutions were prepared and loaded into the instrument:

### Acid Violet Stain (Helena 551758)

Ingredients: The stain is comprised of Acid Violet Stain.

Preparation for Use: Dissolve the dry stain in 1 liter of 10% acetic acid (100 mL acetic acid into 900 mL diH₂O) and mix thoroughly. Fill the SPIFE stain vat.

### Citric Acid Destain (Helena 551959)

Ingredients: After dissolution, the destain contains 0.3% (w/v) citric acid.

Preparation for Use: Pour 11 L of deionized water into the Destain vat. Add the entire package of Destain. Mix well until completely dissolved.

### Tris-Buffered Saline

The powder contains Tris base with Tris-HCl and sodium Chloride. Dissolve the powder in 8 L of deionized water and mix until dissolved.

Albumin, from Bovine Serum (Sigma-Aldrich A7030)

Ingredients: BSA Cohn Fraction V. Lyophilized powder

Preparation for Use: Use as is in preparation of Dilution Solution.

### Dilution Solution; 10% BSA in Saline-NaCl 0.9% W/V Solution

Ingredients: 10% BSA in Saline-Sodium Chloride 0.9% W/V Solution.

Preparation for Use: Transfer and dissolve 5 g BSA in 50 mL Saline-0.9% W/V solution. Mix on magnetic stirrer until completely dissolved; approximately 15 minutes.

The albumin dilution solution was used to dilute samples beyond the apoB linearity of the Lipo-IFE assay. Diluting with this solution maintains the surface tension relationship necessary for appropriate sample deposition.

Quality control samples were prepared from lyophilized serum reconstituted in 1.5 ml of deionized water, swirling gently and incubating in a rocker for 15 minutes. 200 μl aliquots were portioned into tubes. An abnormal and a normal control were run on each gel with QC solutions. For sample preparation, in aliquot tubes, the first two positions were reserved for QC samples.

The sample tray was loaded into an automated plate carrier (Hamilton). The samples were loaded into the SPIFE 3000 sample tray.

Gel Preparation: The required number of Applicator Blades (Helena) were prepared by removing protective guards and positioned on the instrument. The Applicator Blades were loaded onto the instrument and the reagent vial placed into position. 0.5-1 mL REP Prep (Helena) was dispensed onto the electrophoresis chamber floor prior to putting the SPIFE Vis Cholesterol Gels on to the chamber floor. The gel was positioned on alignment pins and electrodes were positioned on the pins to complete the circuit between gel and DC power supply. Using the initial blotter (Helena), the edge of the blotter was lined up with the edge of the Mylar backing and the blotter was aligned between the gel blocks. The blotter was removed from the gel from the side of the gel initially encountering the blotter.

Operation: The cholesterol operation was initiated on the instrument and it was checked that sample loading proceeded correctly. The electrophoresis procedure continued automatically. Before electrophoresis had completed, working pAb was prepared by mixing 1 part anti-apoB sera with 4 parts normal saline. The electrodes, blotters, and blades were removed immediately on completion.

The “Gel Block Remover” was used to scrape off the gel-blocks at the cathodic and anodic ends of the gel. The Rigid Antisera Template (Helena) was placed upon the gel, aligning chamber floor pins with holes in the template. 250 μL of working ApoB antisera was dispensed in each sample lane on the rigid template. The “comb blotter” was placed on the anode port of the Rigid Antisera Template and excess antisera removal is observed; it is allowed to remain in position for at least 30 seconds. The comb blotter was removed and the Rigid Antisera Template is carefully removed from the gel.

A blotter was roll positioned on the gel, minimizing trapped air bubbles, and two more blotters are placed on top. The Rigid Antisera Template was placed upon the chamber floor alignment pins and the gel was press-blot by center-placing a weight upon the rigid template for 60 seconds.

The blotters, weight, and rigid template were removed and the gel was placed into a shallow container-bath of Tris-Buffered Saline (TBS) solution (approximately 50 mL), making sure the agarose side was facing. The wash tray was gently agitated or placed on shaker for 1 minute.

The blotting method was repeated and the gel was placed into the shallow container-bath of TBS solution (approximately 50 mL), making sure the agarose side was facing up. The wash tray was manually agitated or placed on a shaker for 1 minute. The gel was removed from the bath and excess TBS is removed from the gel by gently shaking. The second blotting method was repeated again, and one final time after dispensing approximately 0.5-1 mL REP Prep onto the left side of the electrophoresis chamber.

The electrodes were replaced on the gel and the SPIFE 3000 dry cycle initiated. The automated drying procedure was followed with the automated washing, staining, and destaining procedure on the SPIFE 3000.

The fractions present in the gel lanes were evaluated with the Quick Scan 2000, performing neutral density densitometer scans as directed in the Quick Scan 2000 Operator Manual. Each scan consisted of 3 fractions, Lp(a)-P, vLDL-P, and LDL-P.

Helena densitometer software automatically calculates and prints the relative percent and the mg/dL for each band when the specimen total Apo-B is entered as (% of Fraction)×(total Apo-B). The particle number is calculated in a preferred laboratory information system as (mg/dL)/0.054=PN. Results are reported as Particle Number (PN=nmol/L).

### Example 2

### Lp(a) Subform Size Identification Using Zonal Gel Immuno-Fixation Electrophoresis

The experiments and descriptions thereof that follow demonstrate a relationship between migration velocities and Lp(a) subform size. Faster Lp(a) particles correspond to larger apo(a) apolipoprotein moieties and slower particles indicate smaller apo(a). Additional distinction is drawn from known Lp(a) size standards.

Apo(a) MW's have been measured on 130 samples with migration biases as well as doublets. All results confirm the proportionality between MW and Migration Velocity on zonal gels. Also, it shows that subform types can be blended without compromise to either migration velocity or subform size. Such will allow the preparation of a subform reference control which would establish “on gel” Lp(a) subform MW markers. Such a reference would provide quantitative quality control for particle number and a migration velocity reference to categorize Lp(a) particles into large, mid- and small MW's. All individuals express two subform types. Doublets are seen on the gel when there is sufficient kringle/MW difference between the apo(a) of the Lp(a)-Particles to match the resolving ability of the system. This system is adequate to probe and validate clinical significance of Lp(a)-subforms. The gels can be modified to increase resolution if necessary by methods known in the art. The system identifies both bands for particle number and size.

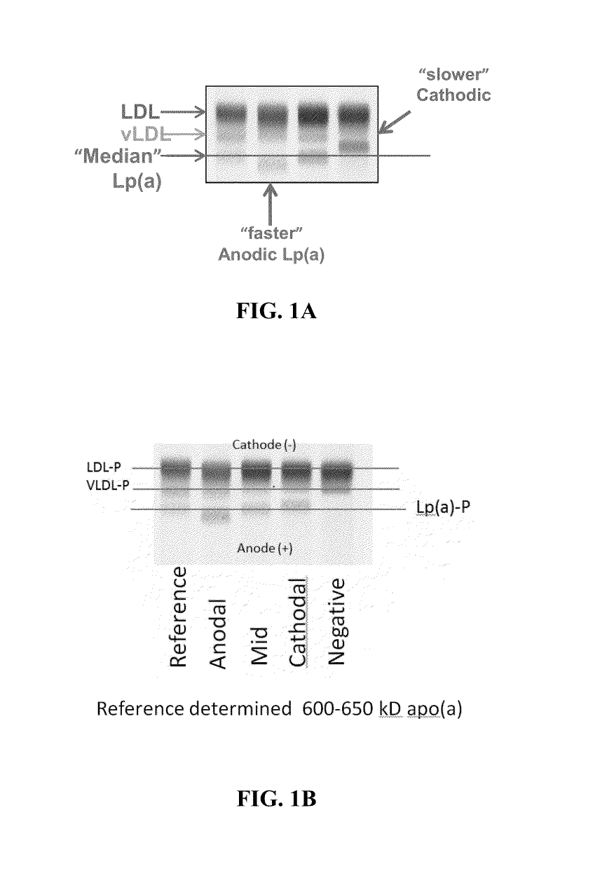

FIGS. 1A and 1B show variable Lp(a)-P migration rates via the results of a Lipo-IFE protocol, as described herein. FIG. 1A provides a basic example gel for evaluating differences in Lp(a) migration. FIG. 1A labels electrophoretic banding on the example gel corresponding to LDL, vLDL, median Lp(a), slower cathodic Lp(a), and faster anodic Lp(a). In FIG. 1B, the results of 5 samples run according to the protocols in this application are shown. Samples all contain LDL and VLDL, and all but the negative control further includes Lp(a) particles. The reference (known) Lp(a) content comprises an apo(a) moiety of 600-650 kD. An “anodal” sample in lane 2 contains Lp(a) with an apo(a) moiety of greater than 650 kD, the “mid” sample in lane 3 contains apo(a) moieties of the same mass as the reference, 600-650 kD, and the “cathodal” sample contains apo(a) moieties of less than about 600 kD. FIGS. 1A and 1B illustrate the principle of differential detection and quantification of Lp(a) subforms.

FIG. 2 shows examples of samples with differential Lp(a)-P migration bias on zonal gels, separated via the Lipo-IFE protocol described herein. In FIG. 2, a series of samples have been run on a gel in parallel on the Lipo-IFE system. The cathode and anode ends of the gel are labeled and solid lines represent the expected position of Lp(a) particles after separation. Four samples have been highlighted (sample numbers 10, 73, 24, and 44). These samples show distinct Lp(a) subform size difference due to the migration rates of smaller subforms (samples 10 and 73, with dashed outline and positioned toward the cathodal end of the substrate) and larger subforms (samples 24 and 44, with a solid outline and positioned toward the anodal end of the substrate).

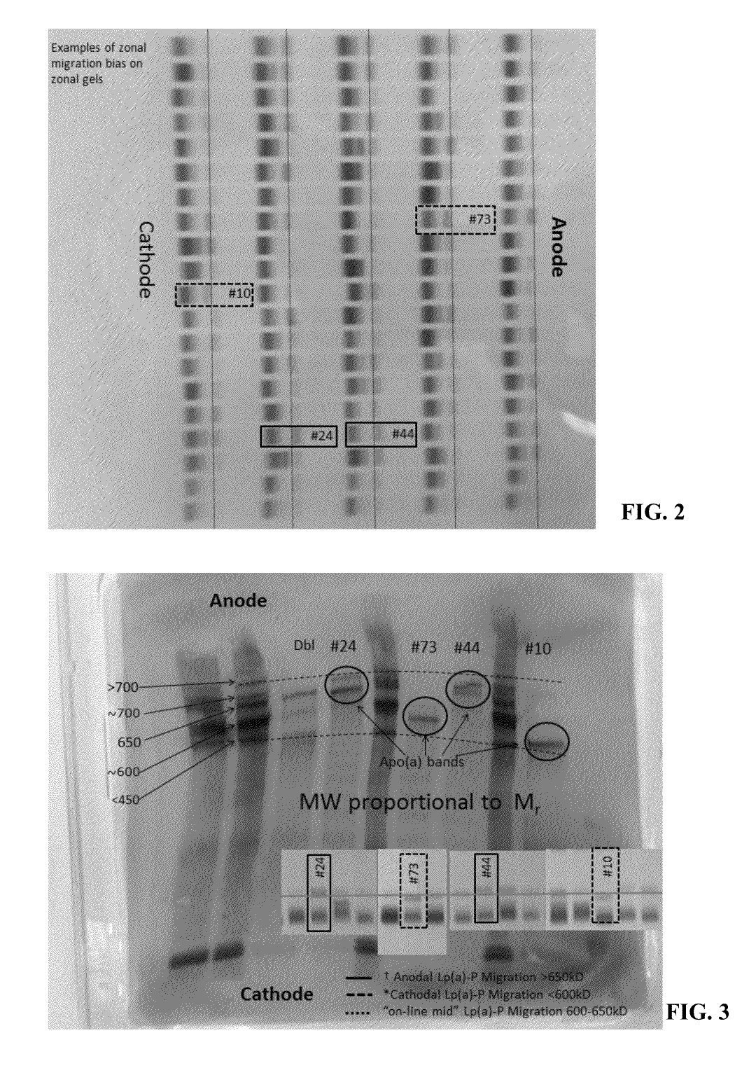

FIG. 3 compares the zonal gel (Lipo-IFE protocol, inset) shown in FIG. 2 to the same samples in a western blot. In FIG. 3, samples 24, 73, 44, and 10 are further analyzed in a western blot analysis after apo(a) removal from the Lp(a) particles. Western Blot analysis was carried out using standard protocols with Apolipoprotein(a) Isoform Analysis (AAISO), using the Novex® WesternBreeze™ Chromogenic Western Blot Immunodetection Kit (Invitrogen Life Technologies). AAISO uses electrophoresis and western blot to measure Apo(a) isoforms in serum or EDTA plasma. Serum (or plasma) is first reduced in dithioerythritol and 6-aminocaproic acid then denatured in beta mercaptoethanol. The denatured sample is then loaded onto a 4% Tris Glycine gel and electrophoresed. After the electrophoresis, the proteins are transferred from the gel to a polyvinylidene fluoride transfer membrane. Western detection is then run on the PVDF membrane. All unoccupied binding sites are blocked with a Hammerstein Casein solution. The membrane is then incubated in a goat anti Lp(a) primary antibody followed by an alkaline phosphatase-conjugated anti IgO secondary antibody. A chromogenic substrate is added for the color development and the membrane is analyzed. Multiple reference standards intersperse the various lanes. Sample 24, which is an anodal (or larger) Lp(a) particle, exhibits a larger separated apo(a) in the western blot, appearing at around 700 kD and greater than 700 kD. Sample 73, which is a cathodal, smaller particle in the zonal gel) corresponds to a smaller apo(a) moiety around 600 kD in the western blot. Samples 44 and 10 repeat the pattern with an anodal Lp(a) similarly having larger apo(a) bands at ˜700 kD and a cathodal Lp(a) having a smaller apo(a) band at less than 450 kD on the western blot.

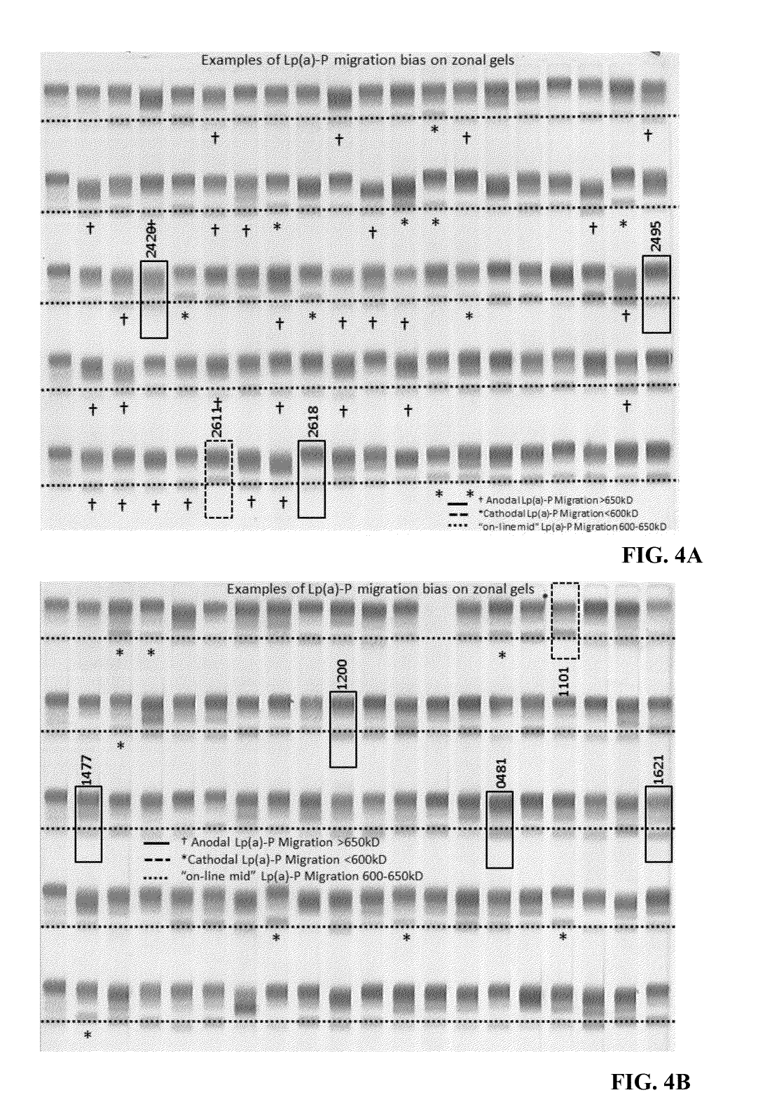

FIGS. 4A and 4B show more examples of the Lp(a)-P migration differentials in a high-throughput run. In FIGS. 4A and 4B, zonal gels run in parallel show variation among patient samples in a high-throughput experiment. Anodal particles of more than about 650 kD and cathodal particles of less than about 600 kD are identified. Anodal samples include sample numbers 1147, 1200, 0481, 1621, 2420, 2495, and 2618. Cathodal samples include sample numbers 1101 and 2611.

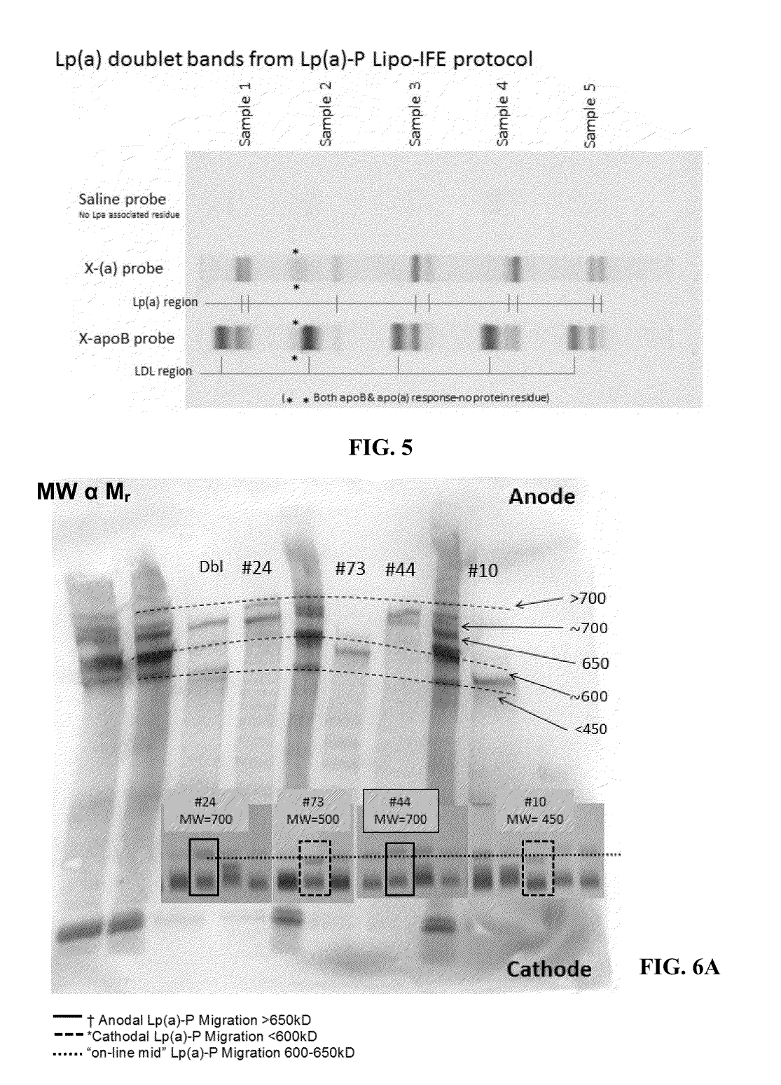

FIG. 5 shows comparison of apo(a) content and apoB content in a series of samples on a zonal gel. In FIG. 5, five samples were probed by anti-apoB sera and anti-apoA sera, showing doublet banding, a product of some subjects having two sizes of apo(a) moieties on their Lp(a) particles. Lp(a) doublet banding is seen on samples 1, 3, 4 & 5; these samples contain both apoB & apo(a) probe response without non-specific protein residue in saline lane. Doublet banding is not artifactual. Lp(a) doublet banding can be equal, primarily cathodal, or anodal with differential resolution. Sample 2 has single Lp(a), but contains both apoB and apo(a) response, cathodal to LDL and without non-specific protein residue in the saline lane. The frequency of doublet banding <1%. Saline probe is used to establish presence of artifactual banding.

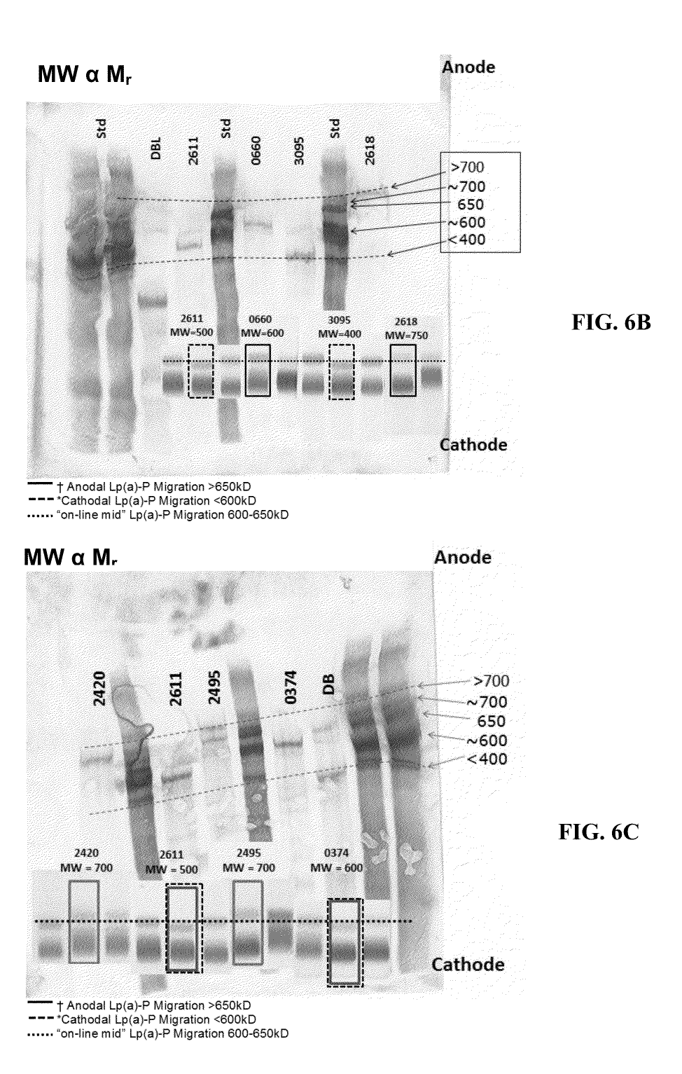

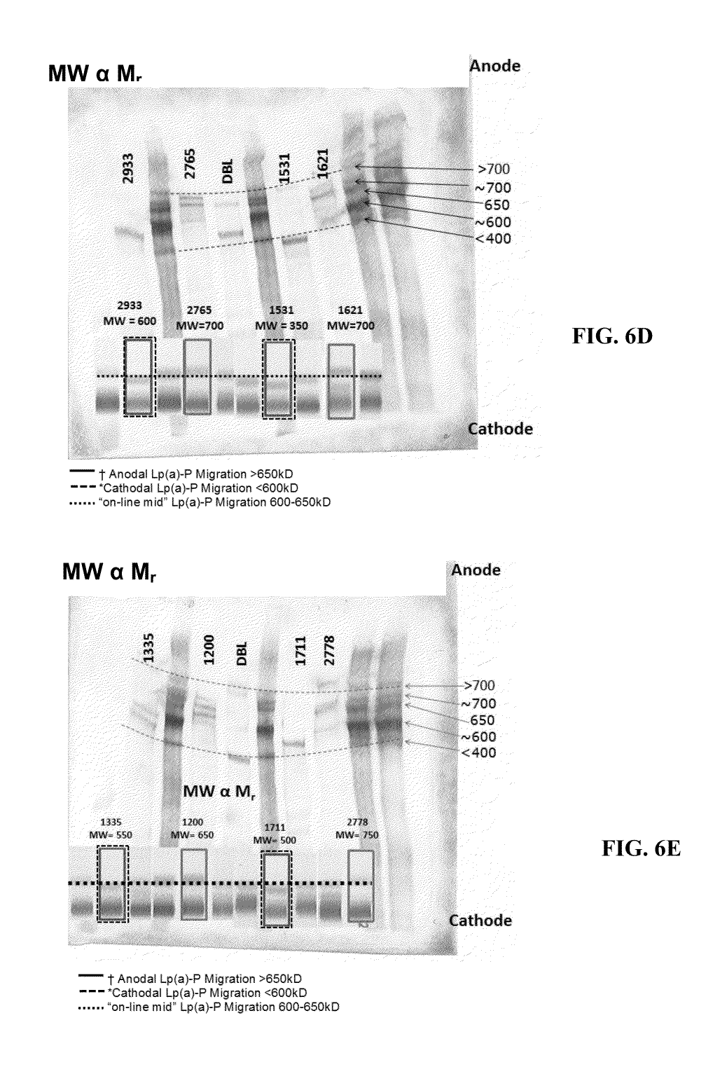

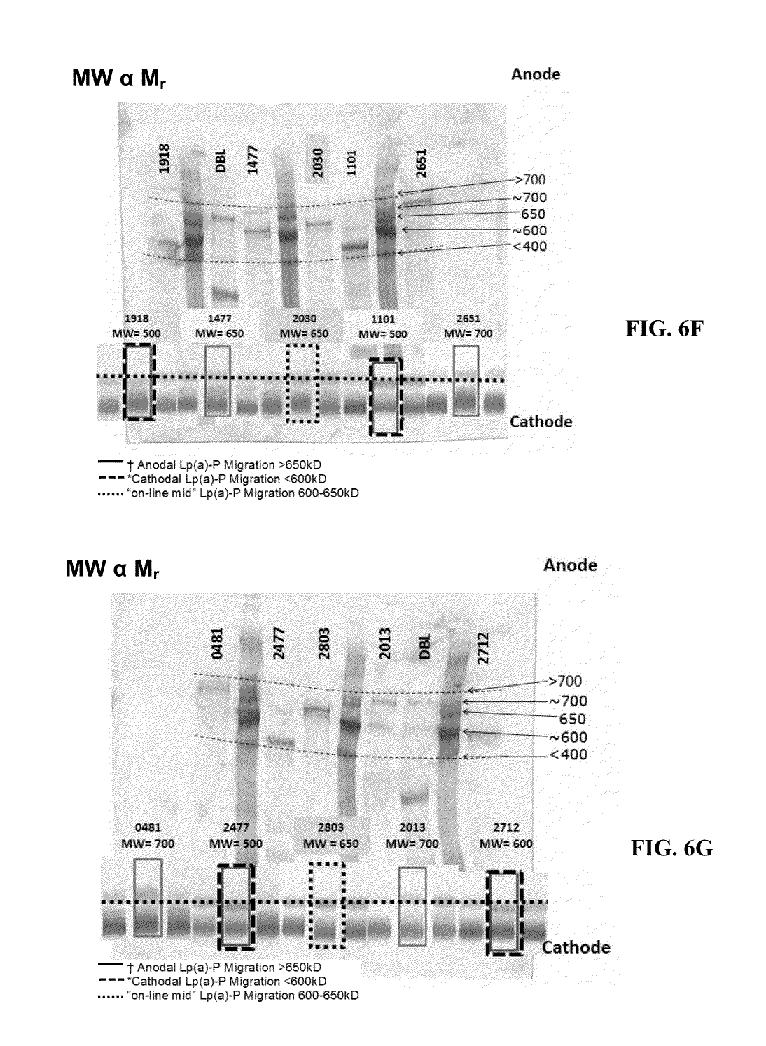

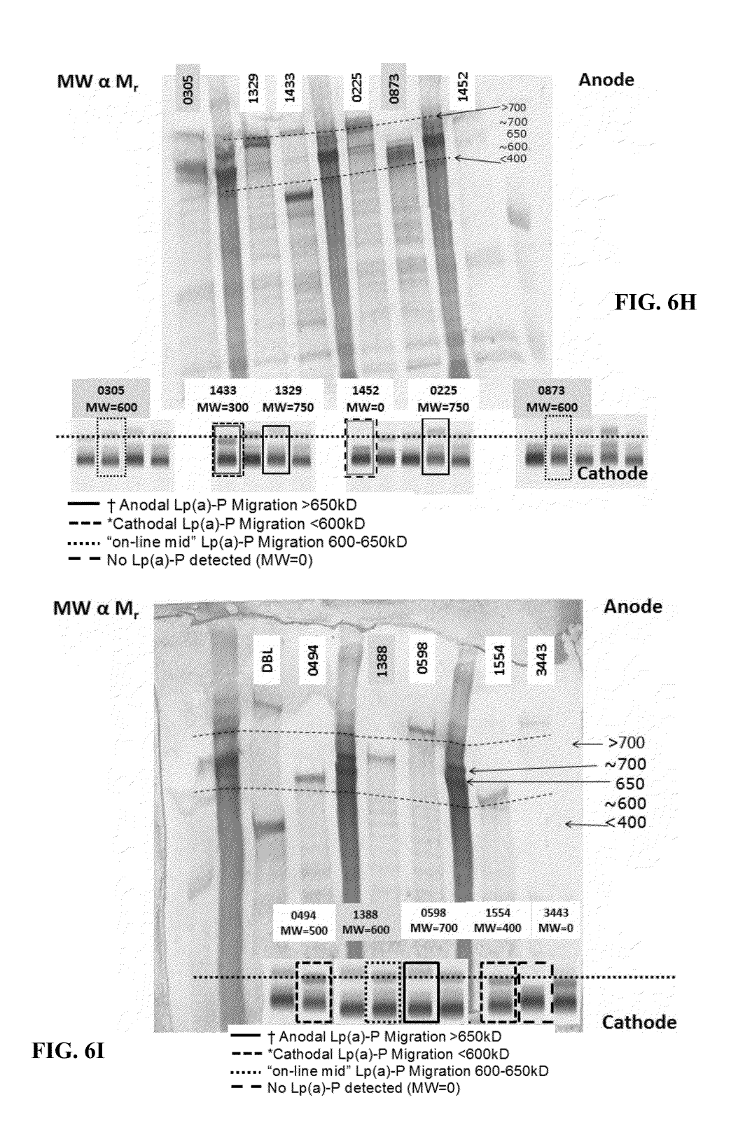

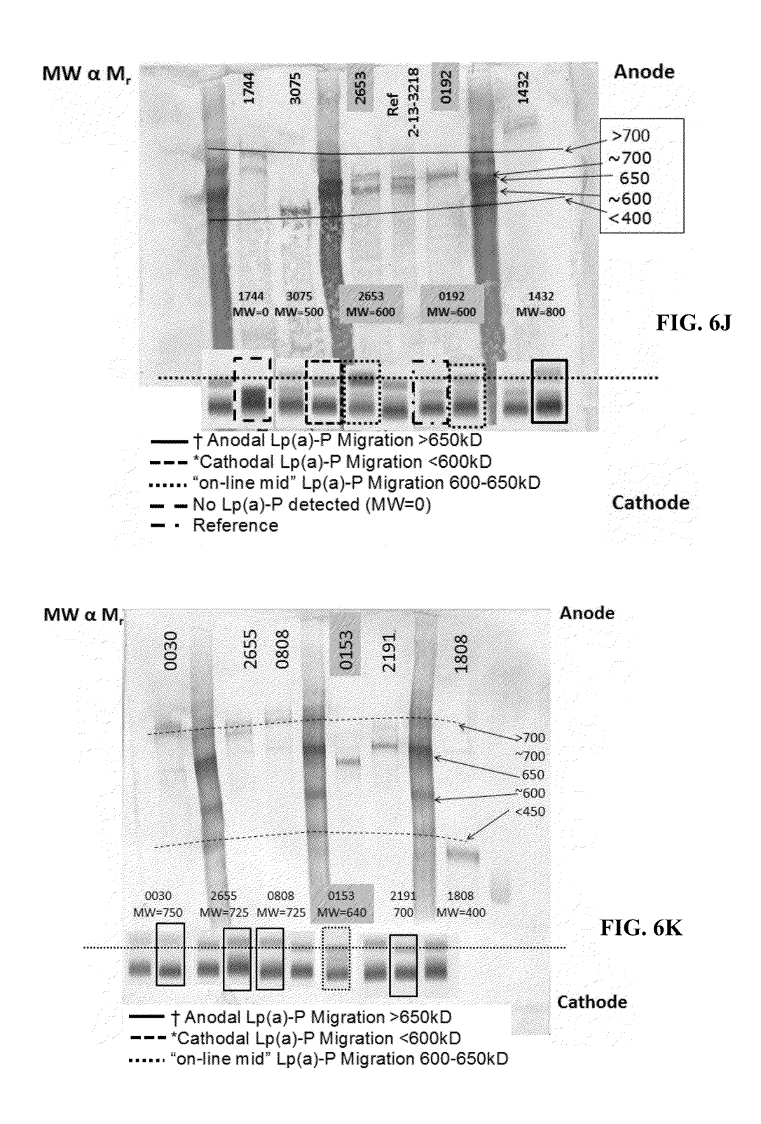

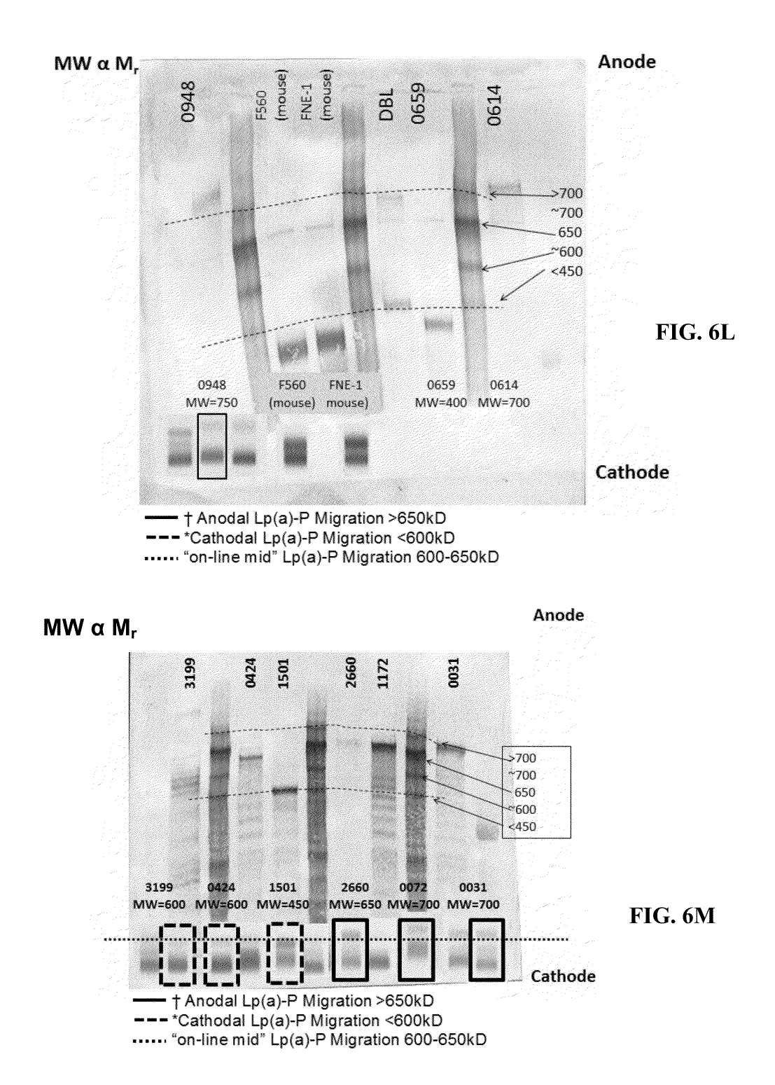

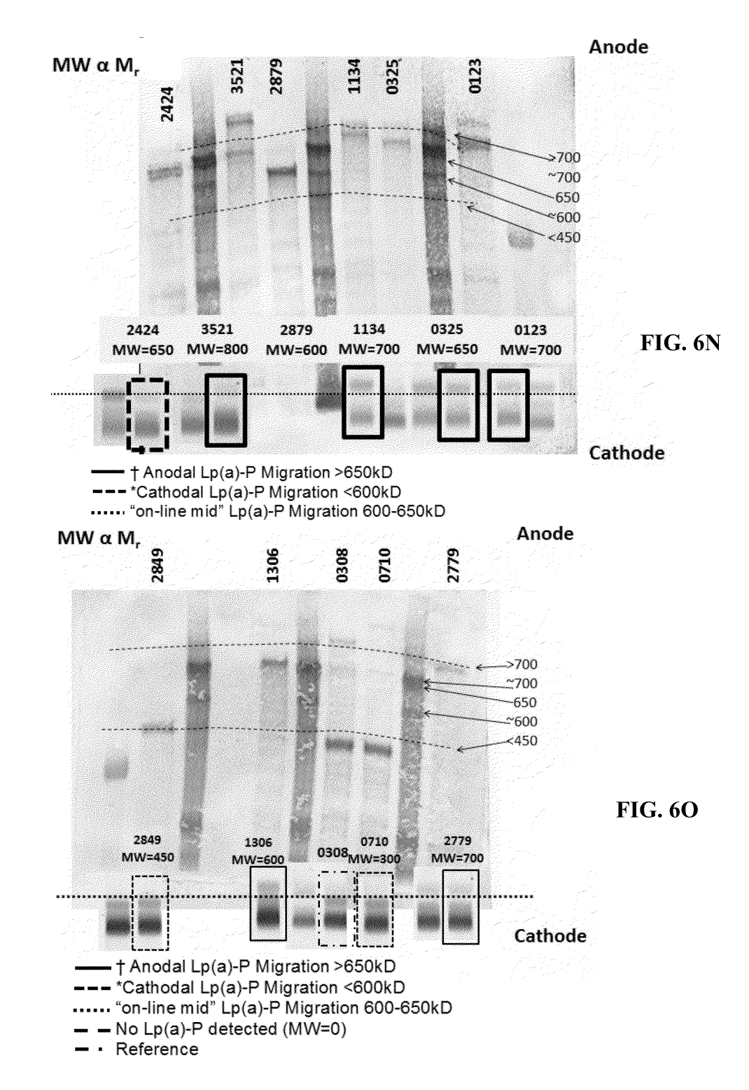

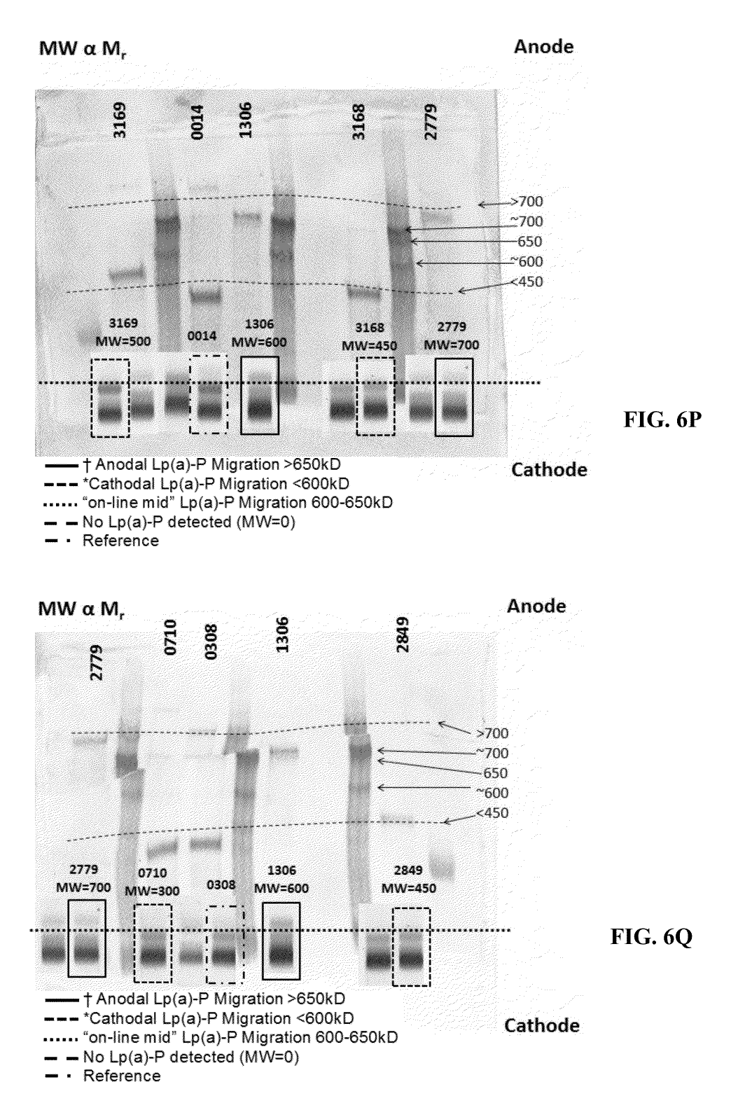

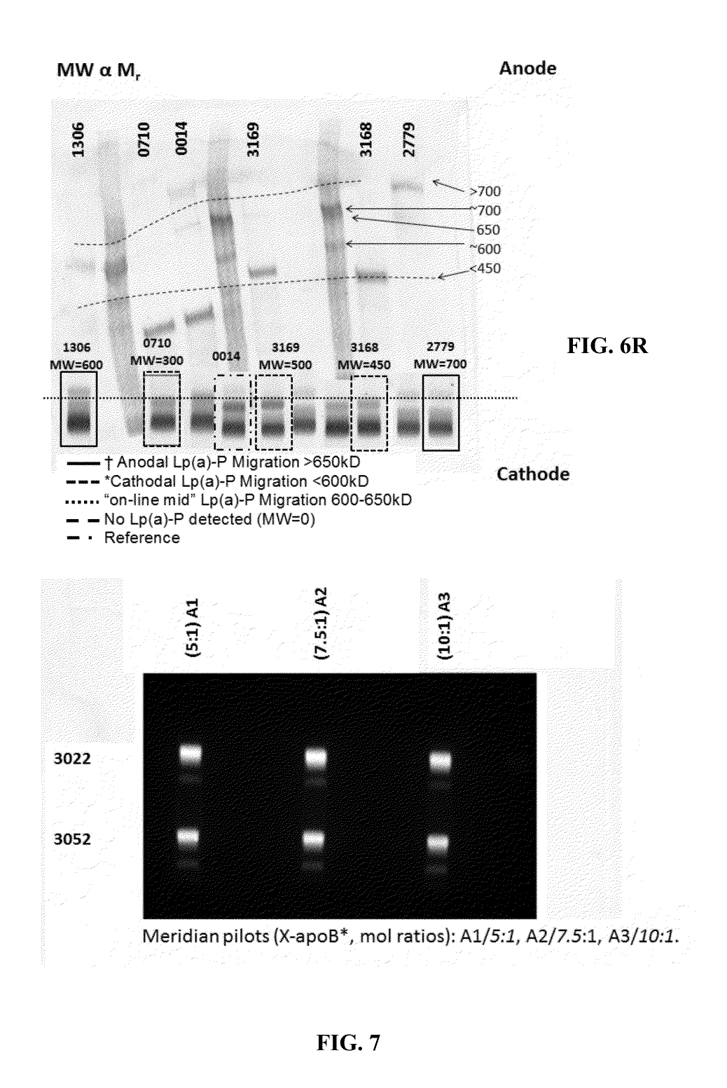

FIGS. 6A-6R present data comparing Lp(a)-P zonal migration velocities (inset, from FIGS. 2, 4A, and 4B) associated with increasing MW of apo(a), measured by western blot. In FIGS. 6A-6R, more than 100 samples with Lp(a) zonal migration biases were compared to apo(a) isoform size analysis by western blot analysis, as described above. The results from 19 experiments reflecting the same setup and analysis as shown in FIG. 3 are presented. The results show consistent agreement of the new zonal gel method for analyzing Lp(a)-P subform size with the more intensive analysis of separated apo(a) moieties from the same particles.

### Example 3

### Validation of Lp(a) Subform Size Identification Using Zonal Gel Immuno-Fixation Electrophoresis

Lp(a) subform size identification using zonal gel immuno-fixation electrophoresis was validated using an assay employing Meridian Custom conjugated Anti-apoB100*-A1exa488 antibodies.

Custom conjugated antibodies were prepared and examined to show the distinction between apoB-containing particles. HDL production Calbiochem Anti-apoB was purified and conjugated to A1exa488 fluorophore. Three 500 uL pilot lots were prepared to study signal:noise ratios. Lots were prepared where lot A1 contained a 5:1 dye to polyclonal antibody ratio, lot A2 contained a 7.5:1 ratio, and lot A3 contained a 10:1 ratio. All Concentrations were normalized to antibody titer for existing working Calbiochem antisera. Serums were chosen with variable Lp(a), VLDL, and LDL concentrations. Doublet samples, with confirmed large and small Lp(a)-P, were assayed with non-specific protein Acid Violet and apoB-specific antibody to provide insight on subform dependence of the assays. The optical density of each fluorescent label, corresponding to an antibody on Lp(a), LDL, or VLDL was measured in a densitometer. All results of lipoprotein levels are reported in relative percentages.

FIG. 7 shows fluorescent imaging for labeled anti-apoB antibodies bound to Lp(a), LDL, and VLDL particles. The image is a 3Blot/TBS-Image (wet) (the dried gel was opaque and unsuitable for imaging). The top row shows sample 3022 at the dilution levels of 5:1 (A1), 7.5:1 (A2), and 10:1 (A3) Alexa Fluor® fluorescent dye:polyclonal antibody, in respective columns. The same experiment is shown for sample 3052 in the bottom row. There are no observable differences in optical properties between the dilution ratios.

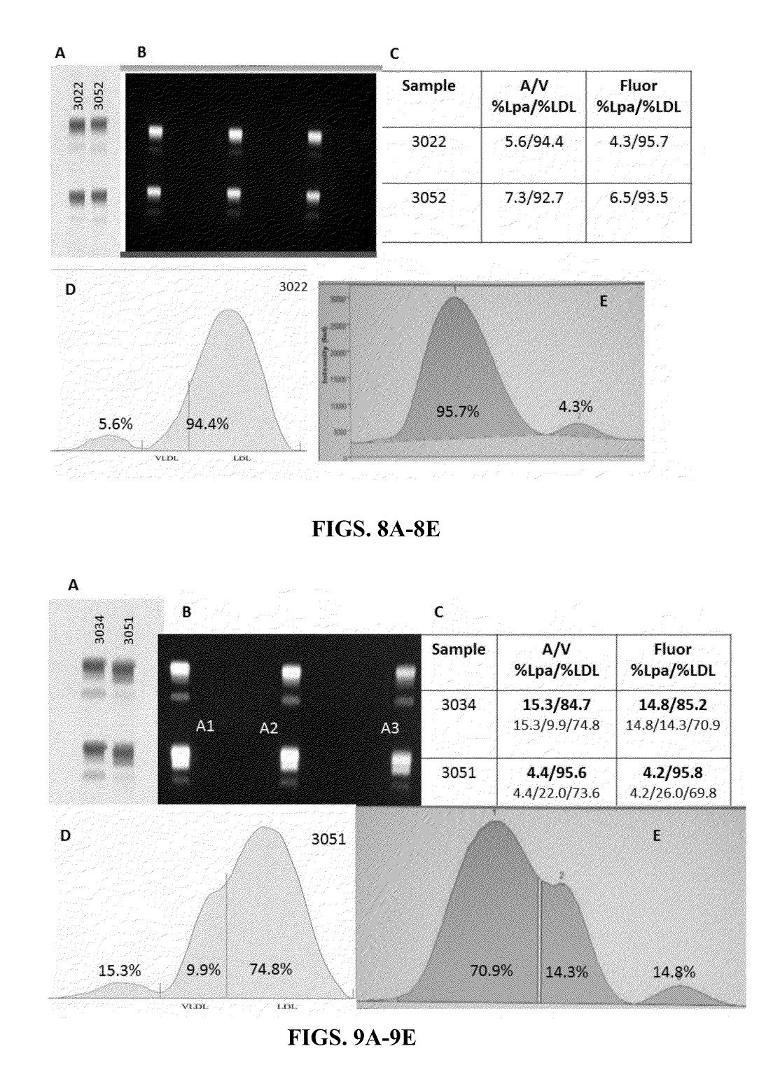

FIGS. 8A-8E show results of an initial comparison of acid violet staining detection methods (A/V) and apoB* fluorescently-tagged antibody labeling methods, for Lp(a)-P distinction from LDL-P in a single sample. FIG. 8A shows a native zonal gel separation of sample numbers 3022 and 3052, in respective columns containing duplicate runs, labeled by acid violet staining. There are 4 total sample runs, where the LDL is evident as a strong band and Lp(a) as a weak band below it. FIG. 8B shows optical imaging with fluorescence of the same samples, where 3022 is on the top row and 3052 is on the bottom row. The columns correspond to different fluorescent dye:anti-apoB ratios in reagents A1, A2, and A3, previously described. FIG. 8C presents numerical results of the optical density readings of each sample, where %Lpa means percent Lp(a) (lipoprotein a) of total detected sample and %LDL means percent LDL (low-density lipoprotein) of the total detected sample. FIG. 8D is the optical density reading of the acid violet staining of sample 3022 in profile and FIG. 8E is the optical density reading of the fluorescent conjugate of sample 3022 in profile (in the reverse direction of the A/V profile).

FIGS. 9A-9E show results of an initial comparison of acid violet staining detection methods (A/V) and apoB* fluorescently-tagged antibody labeling methods, for Lp(a)-P distinction from LDL and VLDL in a single sample. FIG. 9A shows a native zonal gel separation of samples 3034 and 3051, in respective columns containing duplicate runs, labeled by acid violet staining. There are 4 total sample runs, where the LDL is evident as a strong band and Lp(a) as a weak band below it. FIG. 9B shows optical imaging with fluorescence of the same samples, where 3034 is on the top row and 3051 is on the bottom row. The columns correspond to different fluorescent dye:anti-apoB ratios in reagents A1, A2, and A3, previously described. FIG. 9C presents numerical results of the optical density readings of each sample, where %Lpa means percent Lp(a) (lipoprotein a) of total detected sample and %LDL means percent LDL (low-density lipoprotein) of the total detected sample. Further detailed analysis show the levels of Lp(a), VLDL, and LDL below the Lp(a)/LDL levels. FIG. 9D is the optical density reading of the acid violet staining of sample 3051 in profile and FIG. 9E is the optical density reading of the fluorescent conjugate of sample 3051 in profile (in the reverse direction of the A/V profile).

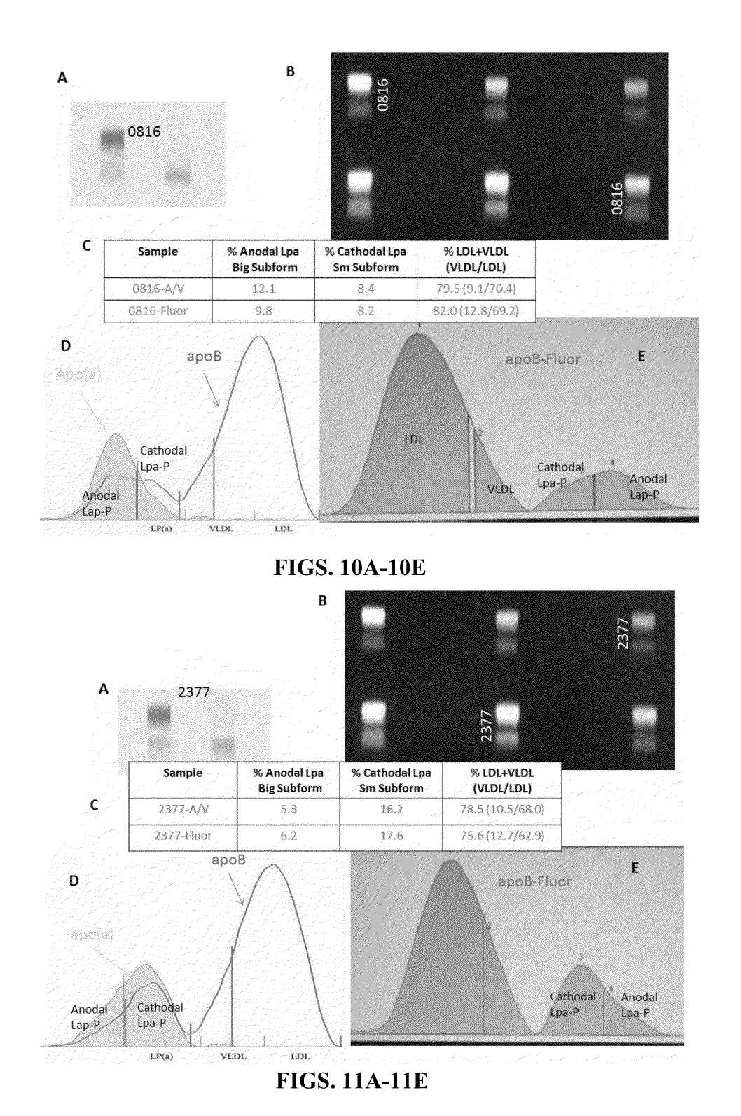

FIGS. 10A-10E show results from a sample comparing the distinction in anodal (large) Lp(a) subforms and cathodal (small) Lp(a) subforms with acid violet staining detection methods (A/V) and apoB* fluorescently-tagged antibody labeling methods, for Lp(a)-P distinction from LDL and VLDL in a single sample. Both LDL, VLDL and Lp(a) were probed with anti-apoB-488*. The probe on the right of the “A” figures was an anti-apo(a) with A/V staining; this probe confirms the identity of the band as an Lp(a)-P. The data compares the specific apoB* with the non-specific A/V and demonstrates the equivalence of both staining methods and addresses concerns re non-specific protein staining of A/V. Both techniques used the same antibody: only the reporters were different, apoB-A/V v apoB-488*. FIG. 10A shows a native zonal gel separation of sample 0816, in the left column, labeled by acid violet staining. On the left, anti-apoB antibodies are used for labeling, which are found on all of Lp(a), LDL, and VLDL. On the right, anti-apo(a) antibodies are used, which only label the Lp(a) particles. FIG. 10B shows optical imaging with fluorescence of the same sample where labeled. The columns correspond to different fluorescent dye:anti-apoB ratios in reagents A1, A2, and A3, previously described. FIG. 10C presents numerical results of the optical density readings of each sample, where %Lpa means percent Lp(a) (lipoprotein a) of total detected sample and %LDL means percent LDL (low-density lipoprotein) of the total detected sample. Further detailed analysis show the levels of Lp(a), VLDL, and LDL below the Lp(a)/LDL levels. FIG. 10D is the optical density reading of the acid violet staining of sample 0816 in profile and FIG. 10E is the optical density reading of the fluorescent conjugate of sample 0816 in profile (in the reverse direction of the A/V profile).

FIGS. 11A-11E show results from a sample comparing the distinction in anodal (large) Lp(a) subforms and cathodal (small) Lp(a) subforms with acid violet staining detection methods (A/V) and apoB* fluorescently-tagged antibody labeling methods, for Lp(a)-P distinction from LDL and VLDL in a single sample. The distinction in data is the same as for FIG. 10. FIG. 11A shows a native zonal gel separation of sample 2377, in the left column, labeled by acid violet staining On the left, anti-apoB antibodies are used for labeling, which are found on all of Lp(a), LDL, and VLDL. On the right, anti-apo(a) antibodies are used, which only label the Lp(a) particles. FIG. 11B shows optical imaging with fluorescence of the same sample where labeled. The columns correspond to different fluorescent dye:anti-apoB ratios in reagents A1, A2, and A3, previously described. FIG. 11C presents numerical results of the optical density readings of each sample, where %Lpa means percent Lp(a) (lipoprotein a) of total detected sample and %LDL means percent LDL (low-density lipoprotein) of the total detected sample. Further detailed analysis show the levels of Lp(a), VLDL, and LDL below the Lp(a)/LDL levels. FIG. 11D is the optical density reading of the acid violet staining of sample 2377 in profile and FIG. 11E is the optical density reading of the fluorescent conjugate of sample 2377 in profile (shown in reverse sequence from A/V).

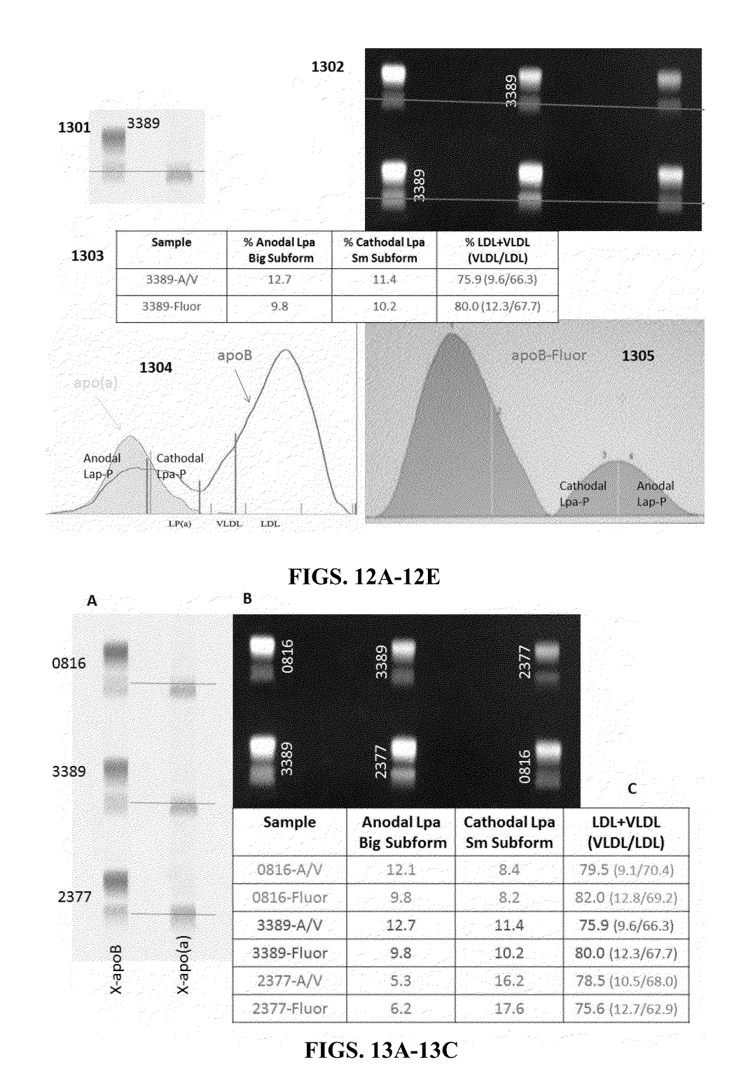

FIG. 12A-12E show results from a sample comparing the distinction in anodal (large) Lp(a) subforms and cathodal (small) Lp(a) subforms with acid violet staining detection methods (A/V) and apoB* fluorescently-tagged antibody labeling methods, for Lp(a)-P distinction from LDL and VLDL in a single sample. The distinction in data is the same as for FIG. 10. FIG. 12A shows a native zonal gel separation of sample 3389, in the left column, labeled by acid violet staining On the left, anti-apoB antibodies are used for labeling, which are found on all of Lp(a), LDL, and VLDL. On the right, anti-apo(a) antibodies are used, which only label the Lp(a) particles. FIG. 12B shows optical imaging with fluorescence of the same sample where labeled. The columns correspond to different fluorescent dye:anti-apoB ratios in reagents A1, A2, and A3, previously described. FIG. 12C presents numerical results of the optical density readings of each sample, where %Lpa means percent Lp(a) (lipoprotein a) of total detected sample and %LDL means percent LDL (low-density lipoprotein) of the total detected sample. Further detailed analysis show the levels of Lp(a), VLDL, and LDL below the Lp(a)/LDL levels. FIG. 12D is the optical density reading of the acid violet staining of sample 3309 in profile and FIG. 12E is the optical density reading of the fluorescent conjugate of sample 3389 in profile (shown in reverse sequence from A/V).

FIGS. 13A-13C shows a summary of FIGS. 10-12, comparing doublet-containing samples 0816, 3389, and 2377 to each other on adjacent gels. FIG. 13A shows the zonal gels labeled with acid violet stain for each sample, with apoB labeled in the first column and apo(a) labeled in the second column. FIG. 13B shows the fluorescence detection for each sample, with the samples clearly labeled. FIG. 13C presents the table summarizing each sample and its relative proportion of anodal (large) Lp(a) subform and cathodal (small) Lp(a) subform along with the LDL and VLDL portions of the sample. The methods show good agreement in calculated ratios of each lipoprotein type level.

These studies validate the results achieved with the Lipo-IFE assay, as described herein.

It will be appreciated that variants of aspects illustrated herein and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the claims below.

What is claimed is:

1. A method for determining the composition of individual Lp(a) subforms in a test sample, the method comprising:
(a) providing a test sample comprising Lp(a) subforms obtained from a subject;
(b) separating the Lp(a) subforms in the test sample along an electrophoretic gel;
(c) measuring the migration velocity of the individual Lp(a) subforms along the electrophoretic gel;
(d) comparing, based on said measuring, the migration velocity of the individual Lp(a) subforms to a reference value; and
(e) determining, based on said comparing, the molar mass of the individual Lp(a) subforms.

2. The method according to claim 1 further comprising:
(f) characterizing, based on said determining, at least two Lp(a) subforms of different molar mass; and
(g) determining, based on said characterization, an Lp(a) subform size distribution of the characterized Lp(a) subforms.

3. The method according to claim 2, wherein the subject has an existing therapeutic regimen, and the method further comprises a step of modifying the therapeutic regimen based on the determined size distribution.

4. The method according to claim 1 further comprising:
(f) measuring the particle number of the individual Lp(a) subforms;
(g) determining the size distribution of the Lp(a) subforms in the sample based on the determined molar mass and particle number of the individual Lp(a) subforms; and
(h) determining a cardiovascular risk value for the subject based on the determined size distribution of the Lp(a) subforms.

5. The method according to claim 4, wherein (h) comprises assigning subject to one of a low, moderate, or high cardiovascular risk category.

6. The method according to claim 1, wherein the molar mass of apo(a) protein of the individual Lp(a) subforms is greater than 600 kD.

7. The method according to claim 1, wherein the molar mass of apo(a) protein of the individual Lp(a) subforms is greater than 700 kD.

8. The method according to claim 1, wherein the molar mass of apo(a) protein of the individual Lp(a) subforms is between 600 and 700 kD.

9. The method according to claim 1, wherein the molar mass of apo(a) protein of the individual Lp(a) subforms is less than 600 kD.

10. The method according to claim 1, wherein said separating comprises separating the Lp(a) subforms in the test sample along a first lane of the electrophoretic gel and said comparing comprises comparing the migration velocity of the individual Lp(a) subforms to the reference value of a control Lp(a) subform sample that is separated along a second lane of the electrophoretic gel.

11. The method according to claim 1, wherein said determining comprises assigning the individual Lp(a) subforms to a low, mid, or high molar mass category.

12. The method according to claim 11, wherein individual Lp(a) subforms having a molar mass less than about 600 kD are assigned to the low molar mass category, individual Lp(a) subforms having a molar mass of between about 600 kD and 700 kD are assigned to the mid molar mass category, and individual Lp(a) subforms having a molar mass of greater than about 700 kD are assigned to a high molar mass category.

13. The method according to claim 1, wherein the separated individual Lp(a) subforms are fixed within the gel prior to said detecting.

14. The method according to claim 1, wherein said measuring comprises:
contacting the electrophoretic gel with a protein dye that dyes the individual Lp(a) subforms; and
detecting the dyed Lp(a) subforms.

15. The method according to claim 14, wherein the protein dye is acid violet.

16. The method according to claim 1, wherein method does not involve use of fluorescence.

17. The method according to claim 1, wherein the individual Lp(a) subforms of the test sample are each bound to a signal-producing molecule capable of producing or causing production of a detectable signal.

18. The method according to claim 17, wherein the method further comprises:
contacting the separated individual Lp(a) subforms of the test sample bound to the signal-producing molecule with a reagent capable of interacting with the signal-producing molecule, wherein the signal-producing molecule produces the detectable signal upon contact with the reagent and wherein said method further comprises detecting the detectable signal.

19. The method according to claim 17, wherein the individual Lp(a) subforms of the test sample are bound to signal-producing molecules that are distinguishable from one another.

20. The method according to claim 17, wherein the detectable signal is detectable by radiometric, colorimetric, luminometric, or fluorometric means.

21. The method according to claim 4 further comprising:
(i) selecting a therapy regimen based on the results of said cardiovascular risk value determination.

22. The method according to claim 21, wherein the selected therapy regimen comprises administering drugs and/or supplements.

23. The method according to claim 21, wherein the selected therapy regimen comprises administering a drug selected from the group consisting of niacin, an anti-inflammatory agent, an antithrombotic agent, an anti-platelet agent, a fibrinolytic agent, a lipid reducing agent, a direct thrombin inhibitor, a glycoprotein IIb/IIIa receptor inhibitor, an agent that binds to cellular adhesion molecules and inhibits the ability of white blood cells to attach to such molecules, a calcium channel blocker, a beta-adrenergic receptor blocker, an angiotensin system inhibitor, and combinations thereof.

24. The method according to claim 21, wherein the selected therapy regimen comprises administering a drug selected from the group consisting of niacin, fenofibrate, estrogen, and raloxifene.

25. The method according to claim 21, wherein the selected therapy regimen comprises administering niacin, ezetimibe, a statin, or a combination thereof

26. The method according to claim 21, wherein the selected therapy regimen involves giving recommendations on making or maintaining lifestyle choices based on the results of said cardiovascular risk value determination.

27. The method according to claim 26, wherein the lifestyle choices involve changes in diet, changes in exercise, reducing or eliminating smoking, or a combination thereof

28. The method according to claim 1, wherein the electrophoretic gel is a zonal electrophoretic gel.

29. A method for predicting cardiovascular health, comprising:
obtaining a sample comprising Lp(a) subforms from a patient;
separating the Lp(a) subforms in the test sample along an electrophoretic gel;
measuring particle number of individual Lp(a) subforms in the sample;
determining a cardiovascular risk value for the subject based on the measured particle number of the Lp(a) subforms.
